(19) United States
(12) Patent Application Publication
(10) Pub. No.: US 2024/0024362 A1
(43) Pub. Date: Jan. 25, 2024

(54) DONOR HEMATOPOIETIC CELL CHIMERISM AND ORGAN AND TISSUE TRANSPLANTATION AND AUTOIMMUNE TOLERANCE

(71) Applicant: The Board of Trustees of the Leland Stanford Junior University, Stanford, CA (US)

(72) Inventors: Robert Lowsky, Stanford, CA (US); Samuel Strober, Portola Valley, CA (US); Everett Hurteau Meyer, Belmont, CA (US); Kent Jensen, Redwood City, CA (US); Stephan Busque, Stanford, CA (US)

(21) Appl. No.: 18/028,050

(22) PCT Filed: Sep. 28, 2021
(86) PCT No.: PCT/US2021/052346

§ 371 Date: Mar. 23, 2023

Related U.S. Application Data
(60) Provisional application No. 63/085,717, filed on Sep. 30, 2020.

(51) Int. Cl. A61K 35/28; A61N 5/10; A61K 39/00

(52) U.S. Cl. CPC A61K 35/28; A61N 5/10; A61K 39/4611; A61K 2035/124

(57) ABSTRACT

Compositions and methods are provided for the achievement of organ and tissue transplantation and autoimmune tolerance using the infusion of living and/or deceased donor hematopoietic cells. The methods provided herein provide for conditioning with a plurality of doses of total lymphoid irradiation (TLI), and a single, very low dose of TBI (svldTBI), referred to herein as “TLI-svldTBI-ATG” or “TLI-svldTBI” depending on whether ATG is included. The combination of svldTBI and TLI specifically targets non-lymphoid-tissue resident memory immune cells. An in vitro manipulated donor cell composition is provided for use with the conditioning regimen, in which specific ratios of CD34⁺ and other hematopoietic stem cell and precursor cell populations are combined with defined doses of CD3⁺ T cells, and/or purified regulatory T cells (Treg) cells, invariant natural killer (iNK-T) cells, and/or CD8⁺ memory T cells.

### CROSS REFERENCE TO RELATED APPLICATION

The present application claims the benefit of and priority to U.S. Provisional Patent Application No. 63/085,717, filed Sep. 30, 2020, the entire disclosure of which is hereby.

### STATEMENT REGARDING FEDERALLY SPONSORED RESEARCH

This invention was made with Government support contracts A1109565 and HL075462 awarded by the National Institutes of Health. The Government has certain rights in the invention.

### BACKGROUND

Recipients of living and deceased donor organs and tissue (kidneys, liver, lungs, heart, pancreas, islet, bowel and composite tissue transplants) require strict, and lifelong adherence to combinations of immune suppression (IS) medication to prevent immune mediated organ transplantation rejection. The newer IS medication combinations have marginally improved the rate of acute (early) graft rejection. The risk of acute (within the first year of transplant) graft loss due to immune mediated rejection is about 5-15% in almost all instances. Beyond the first year of transplantation, and despite ongoing IS drug combinations there is continued graft loss (at about 5% per year) due to chronic immune mediated rejection. The majority of deceased donor organs, and living donor organs are lost within 8, and 15 years of transplantation, respectively. Long-term survival after graft loss in the case of kidney transplant is poor and even worse for recipients of lung and heart transplants.

It is well established that the IS medications themselves induce and/or are strongly associated with significant medical comorbidities that include but are not limited to chronic allograft vasculopathy, diabetes, infections, cancers, heart disease, hypertension, renal dysfunction, and osteoporosis and osteopenia. For example, once beyond 5 years from heart transplantation, IS-associated malignancies resulting from multi-drug IS combinations account for >20% of the annual deaths.

Currently there are no existing methods to establish persistent mixed chimerism following living related and unrelated donor and deceased donor combined organ and hematopoietic cell transplants. The establishment of persistent mixed donor hematopoietic cell chimerism in organ transplant recipients could result in immune tolerance, and meet these needs. The main limiting features to successful and safe organ transplantation are acute and chronic immune mediated graft rejection, and the medical comorbidities induced by the combinations of IS medications. There is an unmet medical need to eliminate the lifelong requirement of IS medications with their attendant side effects, and to prevent immune mediated rejection of living and deceased donor organ transplants including kidney, liver, heart, lung and bowel transplants.

### SUMMARY

Compositions and methods are provided for the achievement of organ and tissue transplantation and autoimmune tolerance using the infusion of living and/or deceased donor hematopoietic cells.

In an embodiment, an novel method of recipient conditioning is provided. In this unique method, total lymphoid irradiation (TLI) is fractionated over a plurality of doses, e.g. at least about 5, 6, 7, 8, 9, 10 or more doses administered, and combined with a single, very low dose of total body irradiation (svldTBI), referred to herein as “TLI-svldTBI”. The TLI doses can be combined with anti-thymocyte globulin (ATG), which protocol is then referred to as TLI-svldTBI-ATG”. Typically the svldTBI is the final dose of radiation before transplantation. The dose of radiation for the svldTBI is from about 40 to about 140 cGy, from about 50 to about 120 cGy, from about 75 to about 100 cGy.

The combination of svldTBI and TLI-ATG induces recipient immune cell depletion by specifically targeting non-lymphoid-tissue memory immune cells. The svldTBI is administered at a dose too low to create “marrow space”, and too low to induce the toxicities associated with TBI-based recipient regimens used in BMT protocols, e.g. marrow hypoaplasia with severe cytopenias, mucositis, and other GI toxicities. Targeting and depleting non-lymphoid-tissue resident recipient immune cells, without inducing marrow hypoplasia, can result in improved rates of persistent mixed donor hematopoietic cell chimerism and avoids risks of graft-versus-host disease (GVHD). In some embodiments a TLI-svldTBI conditioning regimen is used in combination with a donor cell composition comprising a non-physiologic ratio of donor-derived CD34⁺ and CD3⁺ T cells.

In an embodiment, an in vitro manipulated donor cell composition is provided, in which specific ratios of CD34⁺ and other hematopoietic stem cell and precursor cell populations are combined with defined doses of CD3⁺ T cells, and/or purified regulatory T cells (Treg) cells, invariant natural killer (iNK-T) cells, and/or CD8⁺ memory T cells. The cells may be isolated from living donors, e.g. from peripheral blood. In an embodiment the cells are isolated from hematologic tissues such as bone marrow, spleen, lymph nodes, etc. from deceased donors. The manipulated cell composition finds particular use in combination with a TLI-svldTBI or TLI-svldTBI-ATG conditioning regimen. The manipulated cell composition induces persistent donor cell chimerism without the risk of GVHD. Persistent mixed chimerism of the infused donor cells can enable organ and tissue transplantation tolerance, as well as tolerance in patients with autoimmune diseases.

In the case of living HLA mismatched related and unrelated donors, donor hematopoietic cells can be mobilized using granulocyte colony stimulating factor (G-CSF)+/−mozobil, and the donor will undergo 1 or 2 consecutive days of high volume (>12 liters) blood apheresis to obtain blood mononuclear cells. The apheresis collection(s) will be processed for CD34⁺ cell enrichment using either fluorescence activated cell sorting (FACS) or magnetic activated cell sorting (MACS) as per manufacturer's guidelines.

The CD34⁺ enriched product will be cryopreserved in the standard manner. The pre-freeze CD34⁺ cell purity is at least about ≥70%. The CD34⁺ cell dose will have a pre-freeze value of from about 4 to about 20×10⁶ CD34⁺ cells/kg recipient weight, for example from about 4×10⁶ CD34⁺ cells/kg; from about 10×10⁶ CD34⁺ cells/kg, from about 12×10⁶ CD34⁺ cells/kg, from about 14×10⁶ CD34⁺ cells/kg, from about 16×10⁶ CD34⁺ cells/kg, from about 18×10⁶ CD34⁺ cells/kg, from about 20×10⁶ CD34⁺ cells/kg.

In some cases, the non-CD34⁺ cell fraction following a CD34⁺ enrichment step is used to obtain a defined dose of CD3⁺ T cells, and will be cryopreserved in the usual manner. The pre-freeze dose of CD3⁺ cells is from about 25 to about 100×10⁶ CD3⁺/kg recipient weight, for example from about 25×10⁶ CD3⁺/kg, from about 35×10⁶ CD3⁺/kg, from about 45×10⁶ CD3⁺/kg, from about 50×10⁶ CD3⁺/kg, up to about 100×10⁶ CD3⁺/kg, up to about 90×10⁶ CD3⁺/kg, up to about 80×10⁶ CD3⁺/kg, up to about 70×10⁶ CD3⁺/kg, up to about 60×10⁶ CD3⁺/kg.

In some embodiments, enriched populations of donor derived CD8⁺ memory T cells, which can be defined as CD3⁺/CD8⁺/CD45RA⁻/CD45RO⁺ are provided at a dose of from about 1 to about 12×10⁶ cells/kg, for example from about 1×10⁶ cells/kg, from about 2×10⁶ cells/kg from about 4×10⁶ cells/kg, from about 6×10⁶ cells/kg, to about 12×10⁶ cells/kg, to about 10×10⁶ cells/kg, to about 8×10⁶ cells/kg. The memory cells may be infused from about 0 to about 3 days after the CD34⁺ enriched cell product, for example from about 0 to 3, from about 1-3, from about 2-3 days following the CD34⁺ enriched cell product. In some embodiments the CD8⁺ memory T cells are provided in the place of CD3⁺ cells.

In some embodiments, donor derived Treg cells, which can be defined as CD4⁺CD25⁺FoxP3⁺ enriched by FACS or MACS methods are infused from about 0 to about 4 days after the infusion of donor CD34⁺ enriched cells; at a dose of from about 1 to about 10×10⁶ cells/kg, for example from about 1×10⁶ cells/kg, from about 2×10⁶ cells/kg from about 4×10⁶ cells/kg, from about 6×10⁶ cells/kg, to about 12×10⁶ cells/kg, to about 10×10⁶ cells/kg, to about 8×10⁶ cells/kg. The Treg cells may be infused from about 0 to about 4 days after the CD34⁺ enriched cell product, for example from about 0 to 3, from about 1-3, from about 2-3 days following the CD34⁺ enriched cell product. In some embodiments, the donor Treg cells are combined with donor CD3⁺ T cells at a ratio of Treg:CD3⁺ T cells ranging from 1:50; 1:20, 1:10, 1:5, 1:2, 2:1, to 3:1.

Tissue from deceased donors can be banked for clinical use in patients receiving organ or tissue transplants, which tissue also finds use for transplantation in patients receiving cell therapies for control of refractory and relapsing autoimmune diseases, and regenerative medicine therapies. Tissues of interest include, without limitation, splenic and bone marrow derived hematopoietic stem cells and precursor cell populations, mesenchymal stem cells, stromal cells CD3+ Th1/Th2Th17/Tfh T cells, CD19+ B cells, regulatory T cells (Treg) and invariant natural killer (iNK T cells)

Methods are provided to establish persistent mixed chimerism for donor-recipient pairs in organ transplantation of all degrees of HLA mismatch. The novel conditioning regimen disclosed herein (TLI-svldTBI-ATG) combined with the unique composition of matter of donor CD34⁺, CD3⁺ and/or CD8⁺ memory T cells, and/or Treg cells supports persistent mixed chimerism and protects against GVHD. The attainment of persistent mixed chimerism enables transplant organ tolerance and immunosuppressive drug minimization and/or cessation.

An aspect of the present disclosure is a recipient transplant tolerance conditioning regimen of 9 doses of TLI; and one svldTBI, combined with ATG, to establish persistent mixed chimerism in organ transplantation. The svldTBI dose employs doses of radiation not previously described or considered clinically meaningful and as such represent an non-intuitive disclosure; that svldTBI provides clinically meaningful depletion of tissue-resident host immune cells that resist donor hematopoietic cell engraftment and the establishment of persistent mixed chimerism. Using TLI-svldTBI-ATG recipient conditioning alters and depletes recipient immune cells and facilitates persistent donor cell chimerism in recipients of deceased donor organ transplants of all degrees of HLA mismatch; and of living related or unrelated donor organ transplants. Consequently, far fewer number of CD34+ hematopoietic cells can achieve hematopoietic cell engraftment, relative to TLI only conditioning regimens. Transplant tolerance is achieved for any solid tissue, including without limitation tolerance for recipients of living related and unrelated, and deceased donor organs, e.g. kidney, heart, lungs, liver, bowel, etc., and tissue and composite tissue transplants that include all degrees of HLA mismatch.

In some embodiments, the methods and compositions disclosed herein are utilized in the treatment of a recipient with an autoimmune disorder. The immune system has a critical role in pathogenesis of these diseases, involving, for example, T-, B-, Natural Killer (NK), and Regulatory T (Treg) cells. Conventional disease modifying therapies for the treatment of autoimmune disorders display efficacy yet none “re-set” and “re-store” the immune dysregulation that underlie the disease pathogenesis. The infusion of hematopoietic cell subsets as outlined in the current application will reset and restore the immune dysregulation underlying the autoimmune disease. In some embodiments, recipient conditioning using TLI-svldTBI is administered immediately prior to the cell infusion. This treatment provides immune tolerance in a manner that enables highly efficacious and durable disease control.

In some embodiments, the methods and compositions disclosed herein are utilized for inducing tolerance in patients undergoing regenerative medicine therapy. Organ and tissue loss through aging, disease, and injury motivate the development of therapies that can regenerate tissues and decrease reliance on transplantations. Regenerative medicine applies engineering tissues to promote regeneration, and can restore dysfunctional, diseased, and injured tissues and whole organs. Specifically, the cells subsets obtained from donor spleen and bone marrow can enhance the intrinsic regenerative capacity of the host by altering its environment through cell injections, and in some cases genetically engineered cells, or through immune modulation. Beneficial therapeutic responses are obtained through indirect means, such as secretion of growth factors and interaction with host cells, without significant incorporation of the cells per say into the host or having the transplanted cells form a bulk tissue. The injected/infused cells can restore organ dysfunction due to normal aging, and correct the injured or diseased environment, by altering the extra-cellular matrix (ECM) to improve tissue regeneration via this mechanism.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1 depicts the example of a living donor kidney transplant. The HLA mismatched kidney donor to the recipient undergoes blood apheresis and cellular product is manipulated to give a unique non-physiologic ratio of CD34⁺ and CD3⁺ T cells. In some cases donor Treg and CD8⁺ T memory cells are added also in a unique non-physiologic ratio to the CD34⁺, CD3⁺ T cell product. The product is labeled ‘mismatched living donor hematopoietic cells’ (mmLD-HC) and is cryopreserved. At some point thereafter (weeks to months) the patient undergoes a kidney transplant in the usual manner from the same donor from which mmLD-HC were manufactured. Immediately after kidney transplant the recipient begins the unique transplant conditioning described in the ‘use patient’ consisting of TLI-svldTBI-ATG. Upon completion of TLI-svldTBI-ATG the donor cell inoculum is thawed and the cells infused into a vein. It is expected that the patient has mixed donor cell chimerism persisting beyond 6 months and is without evidence of kidney graft rejection and graft-versus-host disease. The standard post kidney transplant immune suppression medications are slowly weaned over a period of about 12 months. It is expected the recipient will maintain mixed donor cell chimerism that will persist and therefore it is expected that the recipient will completely stop all immune suppression medication and maintain normal graft function without risk of kidney graft rejection.

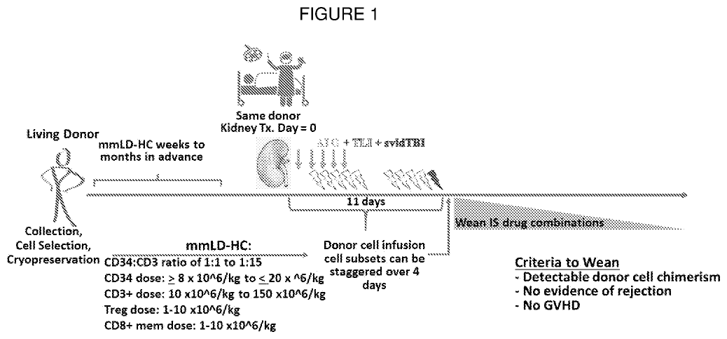

FIG. 2 depicts the example of a deceased donor kidney transplant. The organ procurement team as depicted in this example harvests the donor kidney in the usual manner and also harvest the vertebral column and spleen. The deceased donor kidney is transplanted in the usual manner and soon (within 7 days) thereafter the recipient will begin the unique conditioning of TLI-svldTBI-ATG. While the patient is undergoing the transplant and the TLI-svldTBI-ATG the laboratory is manufacturing the unique cellular product that will induce mixed hematopoietic cell chimerism. Cells obtained from deceased donor vertebral body bone marrow and spleen may have some similar phenotypic profiles to hematopoietic progenitor and immune cells obtained by apheresis from living donors, but are fundamentally physiologically different and as such represent a new composition of matter. Cells as outlined in the composition of matter are manufactured, labeled as deceased donor vertebral body and spleen cells (ddVB+/−SPLN), and are cryopreserved. The ddVB+/−SPLN cell product is thawed upon TLI-svldTBI-ATG conditioning and infused into the recipient vein to establish persistent mixed hematopoietic cell chimerism. It is expected that the patient has mixed donor cell chimerism persisting beyond 12 months and is without evidence of kidney graft rejection and graft-versus-host disease. The standard post kidney transplant immune suppression medications are slowly weaned over a period of about 12 months. It is expected the recipient maintains mixed donor cell chimerism that persists, and therefore the recipient can completely stop all immune suppression medication and maintain normal graft function without risk of kidney graft rejection.

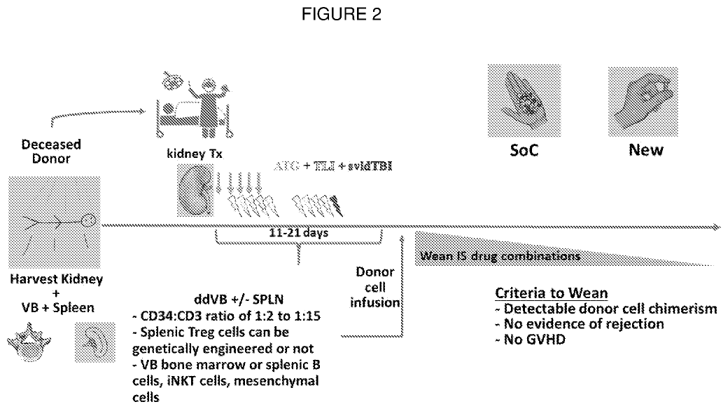

FIG. 3. Defining Hematopoietic Cell Nomenclature.

### DETAILED DESCRIPTION

This invention disclosure describes methods to achieve organ and tissue transplantation and autoimmune tolerance using the infusion of living and/or deceased donor hematopoietic cells. By definition organ and tissue “transplantation tolerance” or “tolerance” refers to normal organ and tissue transplant graft function without the need of immune suppressive medication, and without evidence of organ or tissue graft rejection. The term ‘graft rejection’ encompasses both early (acute) and late (chronic) transplant rejection. In transplant graft rejection, the transplanted tissue is rejected and destroyed by the recipient's immune system.

In some embodiments are provided interdependent components that provide a benefit and promote organ and tissue transplant tolerance for patients receiving organ or tissue transplants, which components can also be applied to patients receiving cell therapies for control of refractory and relapsing autoimmune diseases, including without limitation, Rheumatoid arthritis, Systemic lupus erythematosus (lupus), Inflammatory bowel disease, Multiple sclerosis (MS), Type 1 diabetes mellitus, Guillain-Barre syndrome, Chronic inflammatory demyelinating polyneuropathy, and Psoriasis. The concepts described herein also provide benefits in the field of regenerative medicine.

The term ‘regenerative medicine’ as it applies in this invention disclosure encompasses numerous strategies that include but is not limited to cellular engineering, genetic modification and manipulation of any of the deceased donor splenic or bone marrow derived cell subsets that result in the process of replacing, or regenerating human cells, tissues or organs to restore or establish normal function. It includes the restoration of organ and tissue loss through aging, disease, and injury through, in this case, administered cellular therapies uniquely derived from spleen and bone marrow cells.

While preferred aspects of the present disclosure have been shown and described herein, it is to be understood that the disclosure is not limited to the particular aspects of the disclosure described below, as variations of the particular aspects may be made and still fall within the scope of the appended claims. It is also to be understood that the terminology employed is for the purpose of describing particular aspects of the disclosure, and is not intended to be limiting. Instead, the scope of the present disclosure is established by the appended claims. In this specification and the appended claims, the singular forms “a,” “an” and “the” include plural reference unless the context clearly dictates otherwise.

### Definitions

Where a range of values is provided, it is understood that each intervening value, to the tenth of the unit of the lower limit unless the context clearly dictates otherwise, between the upper and lower limit of that range, and any other stated or intervening value in that stated range, is encompassed within the disclosure provided herein. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the disclosure provided herein.

Unless defined otherwise, all technical and scientific terms used herein have the same meaning as commonly understood to one of ordinary skill in the art to which this disclosure belongs. Although any methods, devices and materials similar or equivalent to those described herein can be used in the practice or testing of the disclosure, the preferred methods, devices and materials are now described.

Hematopoietic cells from deceased sources. There are significant differences in the cellular composition, and immunologic properties of hematopoietic stem cells (HSC) and immune cells obtained from deceased donor (dd) bone marrow (BM) cells compared to the HSC and immune cells obtained by apheresis from healthy living donors.

In the case of deceased donor bone marrow and spleen cells, pure resident bone marrow cells from the VB, pelvis, femur or other bones containing sufficient cells, or from the spleen, are obtained without contamination of cells from the circulation. In the case of apheresis collections, the cellular product is obtained directly from the blood and contains mobilized hematopoietic progenitor cells released from the bone marrow space into the blood. Likewise, the immune cell populations collected through apheresis is different from the populations of immune cells obtained by harvesting deceased donor bone marrow and spleen cells. The cell populations have differing physiologic properties. Table 1 illustrates these differenced.

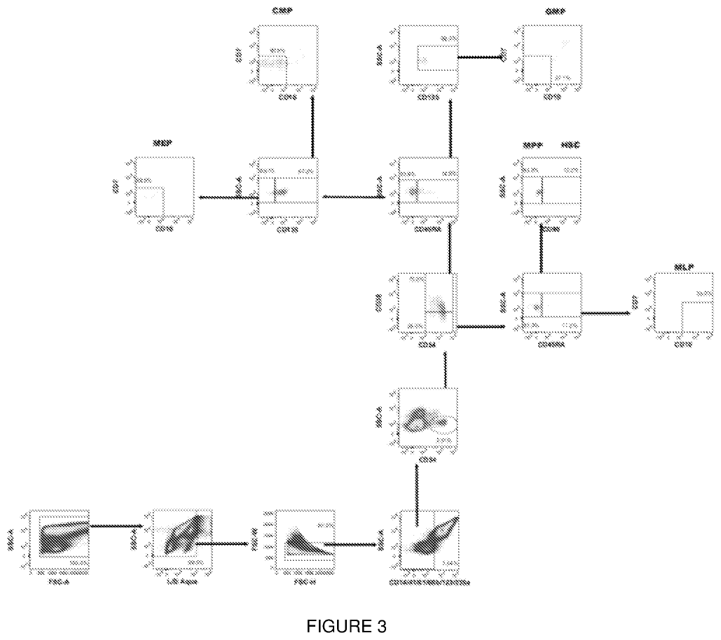

In the case of hematopoietic stem cell populations, the CD34+ cells are at a higher percentage among gated live CD45+ cells in deceased donor vertebral bodies (ddVB) bone marrow (BM) cells compared to products collected by apheresis. The multi-potent, long-term repopulating hematopoietic stem cell (LT-HSC) and the common myeloid progenitor (CMP) are more frequent in ddVB BM cell products. The multipotent progenitor (MPP) and common lymphoid progenitor (CLP) are more common in the living donor apheresis products. See also FIG. 3, which highlights HSC nomenclature.

In the case of immune cell subsets: The CD3+ T cell compartment is much more abundant in apheresis collections. Regulatory and suppressive Treg cells and natural killer (NK) T cells are more common in ddVB BM cell products. These cells suppress GVHD reactions and can enhance donor cell engraftment. The myeloid-derived suppressor cells (MDSCs) are more frequent as a percentage of the nucleated cells in G-mobilized apheresis products compared to their percentage in ddVB BM cells. These immature myeloid cells have regulatory and suppressive qualities that inhibit alloreactive immune responses after organ transplantation and help promote mixed chimerism and organ transplant tolerance (see, for example, Blood Advances 2021: Vol 5, issue 17, 2021: Development of immunosuppressive myeloid cells to induce tolerance in solid organ and hematopoietic cell transplant recipients). The differing cellular composition can highlight why deceased donor products may establish persistent mixed chimerism with fewer numbers of CD34+ cells; there are many more LT-HSC and CMPs and these are critical in promoting long term donor cell engraftment.

TABLE 1
Comparison of Cellular Subsets from Products Obtained by Apheresis
of Living Donors to Products from Deceased Donor Bone Marrow Cells.

| | | G-CSF (G) apheresis | | Deceased donor VB | |
|---|---|---|---|---|---|
| | Specific marker | living donor, mean | | bone marrow, mean | |
| Tested sub-population | of subpopulation | (range) | | (range) | |
| T cells | CD3⁺ (% of CD45⁺) | 46.6 | (22.2; 58.8) | 15.1 | (3.80 21.8) |
| B cells | CD19⁺ (% of CD45⁺) | 6.3 | (2.40; 12.20) | 7.1 | (1.30; 15.00) |
| NK cells | CD56⁺ (% of CD45⁺) | 4.3 | (0.90; 11.00) | 2.8 | (0.30; 5.90) |
| Treg | Treg (% of CD45⁺) | 0.8 | (0.04; 2.23) | 4.0 | (1.1; 6.9) |
| CD45RA⁺ Treg | CD45RA⁺ Treg | 0.10 | (0; 0.46) | 1.1 | (0.08; 0.71) |
| | (% of CD45⁺) | | | | |
| CD45R0⁺ Treg | CD45R0⁺ Treg | 0.83 | (0.03; 7.35) | 2.7 | (0.7; 3.1) |
| | (% of CD45⁺) | | | | |
| NK T cells | CD3⁺CD161⁺ T cells | 0.72 | (0.05; 1.2) | 3.4 | (2.1; 5.4) |
| CD34⁺ hematopoietic | CD34⁺ (% of CD45⁺) | 0.19 | (0.08; 0.44) | 2.8 | (1.4; 3.6) |
| progenitor cells | | | | | |
| CD34⁺CD38⁻ CD90⁺ | CD34⁺CD38⁻CD90⁺ | 6.0 | (1.27; 14.56) | 22.4 | (15.6; 30.4) |
| LT-HSC | (% of CD34⁺) | | | | |
| CD34⁺CD38⁻CD90⁻ | CD34⁺CD38⁻CD90⁻ | 52.30 | (28.3; 80.5) | 20.58 | (8.09; 33.81) |
| MPP cells | (% of CD34⁺) | | | | |
| CD34⁺CD38⁻ CD90⁻ | CD34⁺CD38⁺ | 5.4 | (2.7; 8.9) | 2.7 | (1.3; 4.1) |
| CD45RA⁺ CLP cells | (% of CD34⁺) | | | | |
| CD34⁺CD38⁺CD45RA⁻ | CD34⁺CD38⁺CD45RA⁻ | 6.6 | (1.34; 16.34) | 67.18 | (34.01; 80.10) |
| CD135⁺CD7⁻CD10⁻ | CD135⁺CD7⁻CD10⁻ | | | | |
| CMP cells | (% of CD34⁺) | | | | |
| CD14⁺ cells | CD14⁺ (% of CD45⁺) | 28.45 | (13.60; 40.60) | 16.8 | (11.0; 26.3) |
| Total MDSCs | CD14⁺ HLA-DR⁻ | 12.8 | (5.2; 21.5) | 1.6 | (0.80; 3.9) |
| | (% of CD45⁺) | | | | |

By definition organ and tissue “transplantation tolerance” or “tolerance” refers to normal organ and tissue transplant graft function without the need of immune suppressive medication, and without evidence of organ or tissue graft rejection. The term graft rejection encompasses both early (acute) and late (chronic) transplant rejection. In transplant rejection, the transplanted tissue is rejected and destroyed by the recipient's immune system.

Conventional (>15 years) recipient conditioning regimens used in blood and marrow transplantation (BMT) to cure cancer patients are a) total lymphoid irradiation (TLI) combined with anti-thymocyte globulin (ATG), called TLI-ATG, and b) Total body irradiation (TBI) with or without the addition of chemotherapy. These regimens deplete recipient bone marrow stem cell niches and immune cells to create bone marrow ‘space’ and prevent recipient immune mediated rejection of the infused donor cell inoculum, respectively, and thereby enable complete conversion from recipient to donor type hematopoietic cells. The goal in BMT for cancer patients is the establishment of complete donor cell chimerism, as complete chimerism is associated with beneficial graft-versus-tumor (GVT) reactions that aid in cancer cures.

IS drug minimization is defined as maintenance low therapeutic dose single agent IS monotherapy, and is not associated with the medical co-morbidities caused by conventional multi-IS drug regimens. The use of IS drugs and biologic disease modifying drugs for immune suppression is associated with a risk of developing significant medical co-morbidities (serious infections including tuberculosis, bacterial infections, including sepsis and pneumonia, invasive fungal, viral and other opportunistic infections, progressive multi-focal leukoencephalopathy, lymphoma, cancers, hepatobiliary diseases, congestive heart failure and autoimmune-like disorders).

Mixed chimerism is defined as greater than 1% donor but less than 95% donor DNA in such analysis. Individuals who exhibit mixed chimerism can be further classified according to the evolution of chimerism, where improving mixed chimerism is defined as a continuous increase in the proportion of donor cells over at least a 6-month period. Stable mixed chimerism is defined as fluctuations in the percentage of recipient cells over time, without complete loss of donor cells. Candidates for withdrawal of immunosuppression have mixed chimerism until at least 6 months post-transplantation.

The methods and compositions disclosed herein provide for a high level of mixed chimerism, which may be defined as having blood cells that are at least about 20% donor type, at least about 25%, at least about 30%, at least about 35%, at least about 40%, at least about 45%, at least about 50%, or more. The mixed chimerism is stable, i.e. providing for at least about 20% donor blood cells for a period of at least about 6 months, at least about 9 months, at least about 12 months, at least about 18 months, at least about 2 years, or more.

“Major histocompatibility complex antigens” (“MHC”, also called “human leukocyte antigens”, HLA) are protein molecules expressed on the surface of cells that confer a unique antigenic identity to these cells. MHC/HLA antigens are target molecules that are recognized by T-cells and natural killer (NK) cells as being derived from the same source of hematopoietic stem cells as the immune effector cells (“self”) or as being derived from another source of hematopoietic reconstituting cells (“non-self”). Two main classes of HLA antigens are recognized: HLA class I and HLA class II. HLA class I antigens (A, B, and C in humans) render each cell recognizable as “self,” whereas HLA class II antigens (DR, DP, and DQ in humans) are involved in reactions between lymphocytes and antigen presenting cells. Both have been implicated in the rejection of transplanted organs.

An important aspect of the HLA gene system is its polymorphism. Each gene, MHC class I (A, B and C) and MHC class II (DP, DQ and DR) exists in different alleles. HLA alleles are designated by numbers and subscripts. For example, two unrelated individuals may carry class I HLA-B, genes B5, and Bw41, respectively. Allelic gene products differ in one or more amino acids in the α and/or β domain(s). Large panels of specific antibodies or nucleic acid reagents are used to type HLA haplotypes of individuals, using leukocytes that express class I and class II molecules. The genes most important for HLA typing are the eight high expression alleles: MHC Class I and Class II proteins, two alleles for each of HLA-A; HLA-B, HLA-C and HLA-DR.

The HLA genes are clustered in a “super-locus” present on chromosome position 6p21, which encodes the six classical transplantation HLA genes and at least 132 protein coding genes that have important roles in the regulation of the immune system as well as some other fundamental molecular and cellular processes. The complete locus measures roughly 3.6 Mb, with at least 224 gene loci. One effect of this clustering is that “haplotypes”, i.e. the set of alleles present on a single chromosome, which is inherited from one parent, tend to be inherited as a group. The set of alleles inherited from each parent forms a haplotype, in which some alleles tend to be associated together. Identifying a patient's haplotypes can help predict the probability of finding matching donors and assist in developing a search strategy, because some alleles and haplotypes are more common than others and they are distributed at different frequencies in different racial and ethnic groups.

As used herein, the term “HLA matched” refers to a donor recipient pair in which none of the HLA antigens are mismatched between the donor and recipient. HLA matched (i.e., where all of the 8 alleles are matched) donor/recipient pairs have a decreased risk of graft v. host disease (GVHD) relative to mismatched pairs (i.e. where at least one of the 8 alleles is mismatched).

As used herein, the term “HLA mismatched” refers to a donor recipient pair in which at least one HLA antigen, in particular with respect to HLA-A, HLA-B and HLA-DR, is mismatched between the donor and recipient. In some cases, one haplotype is matched and the other is mismatched. This situation is frequently found with organs from living or deceased donors. HLA mismatched donor/recipient pairs have an increased risk of GVHD relative to perfectly matched pairs (i.e. where all 8 alleles are matched).

HLA alleles are typically noted with a variety of levels of detail. Most designations begin with HLA- and the locus name, then * and some (even) number of digits specifying the allele. The first two digits specify a group of alleles. Older typing methodologies often could not completely distinguish alleles and so stopped at this level. The third through fourth digits specify a synonymous allele. Digits five through six denote any synonymous mutations within the coding frame of the gene. The seventh and eighth digits distinguish mutations outside the coding region. Letters such as L, N, Q, or S may follow an allele's designation to specify an expression level or other non-genomic data known about it. Thus, a completely described allele may be up to 9 digits long, not including the HLA-prefix and locus notation.

As used herein, a “recipient” is an individual to whom an organ, tissue or cells from another individual (donor), commonly of the same species, has been transferred. For the purposes of the present disclosure, a recipient and a donor are either HLA-matched or HLA-mismatched.

As used herein, the term “solid organ transplantation” is used in accordance with the conventional meaning of the term, where an organ from a donor, which donor may be living or deceased, in placed into the body of a recipient in the appropriate position and cardiovascular connections to be physiologically integrated into the recipient. Solid organs of interest for transplantation include kidneys, pancreas and including pancreatic islet cells; heart; lungs, intestine, liver, colon, and the like as known in the art. The transplanted organ may be referenced as a “graft”, and the physiological integration of the organ may be referred to as engraftment.

Hematopoietic stem cell transplantation (HCT) is the transplantation of multipotent hematopoietic stem cells, usually derived from bone marrow, peripheral blood, or umbilical cord blood. For the methods of the disclosure, the hematopoietic cells may be engineered into one of two products. The hematopoietic cells are engineered into a product for infusion having a specific pre-determined number of purified (e.g., ≥70% purity) CD34+ progenitor cells and CD3+ T cells. The hematopoietic cells can be obtained from the solid organ donor, and thus are HLA-matched to the solid organ, and HLA-mismatched to the organ recipient. The hematopoietic cells may be obtained from the solid organ donor, and thus are HLA-matched to the solid organ, and HLA-matched to the organ recipient.

Where the donor is deceased, hematopoietic cells may be obtained from bone marrow (e.g. vertebrae, pelvic bone, etc). Where the donor is a living donor, hematopoietic cells may be mobilized (e.g. with G-CSF), and collected by apheresis or similar methods. Alternatively, cells may be obtained from bone marrow (e.g. pelvic bone, etc).

Hematopoietic cells can be frozen (e.g., cryopreserved) for prolonged periods without damaging a significant number of cells. To cryopreserve HSC, a preservative, DMSO, must be added, and the cells must be cooled very slowly in a controlled-rate freezer to prevent osmotic cellular injury during ice crystal formation. HSC may be stored for years in a cryofreezer, which typically uses liquid nitrogen.

“Immunosuppression”, as used herein, refers to the treatment of a graft recipient with agents, primarily to diminish the immune responses of the host immune system against the graft, although the agents may also diminish GVHD of the donor hematopoietic cells. Exemplary immunosuppression regimens are described in more detail herein, but may be conventional for a period of about 6 to 12 months. The recipient is tested for mixed chimerism of the hematopoietic system, and if found to have maintained mixed chimerism after at least 6 months, will be tapered off immunosuppression.

Immunosuppressive treatment of the transplantation patient begins with the induction phase, perioperatively and immediately after transplantation. Maintenance therapy then continues until withdrawal for individuals showing stable mixed chimerism. Induction and maintenance strategies use different medicines at specific doses or at doses adjusted to achieve target therapeutic levels to give the transplantation patient the best hope for long-term graft survival.

Primary immunosuppressive agents include calcineurin inhibitors, which combine with binding proteins to inhibit calcineurin activity, and which include, for example, tacrolimus, cyclosporine A, etc. Levels of both cyclosporine and tacrolimus must be carefully monitored. Initially, levels can be kept in the range of 10-20 ng/mL, but, after 3 months, levels may be kept lower (5-10 ng/mL) to reduce the risk of nephrotoxicity.

Adjuvant agents are usually combined with a calcineurin inhibitor and include steroids, azathioprine, mycophenolate mofetil, and sirolimus. Protocols of interest include a calcineurin inhibitor with mycophenolate mofetil. The use of adjuvant agents allows clinicians to achieve adequate immunosuppression while decreasing the dose and toxicity of individual agents. Mycophenolate mofetil in kidney transplant recipients has assumed an important role in immunosuppression after several clinical trials have shown a markedly decreased prevalence of acute cellular rejection compared with azathioprine and a reduction in 1-year treatment failures.

Antibody-based therapy uses monoclonal (e.g., muromonab-CD3) or polyclonal antibodies or anti-CD25 antibodies (e.g., basiliximab, daclizumab) and is administered in the early posttransplant period (up to 8 wk). Antibody-based therapy allows for avoidance or dose reduction of calcineurin inhibitors, possibly reducing the risk of nephrotoxicity. The adverse effect profile of the polyclonal and monoclonal antibodies limits their use in some patients.

Graft-versus-host disease (GVHD) is an inflammatory disease that is peculiar to transplantation of hematopoietic cells. It is an attack of the donor bone marrow's immune cells against the recipient's tissues. GVHD is a risk for both HLA-matched and -mismatched transplantations. GVHD can occur even if the donor and recipient are HLA-matched because the immune system can still recognize other differences between their tissues. GVHD is usually mediated by T cells, which react to foreign peptides presented on the MHC of the host. The risk of GVHD is markedly reduced in patients with mixed instead of complete chimerism and achieving mixed chimerism is desirable for this reason. In addition, immunodeficiency and infection are more frequently observed in complete versus mixed chimerism.

There are two types of GVHD, acute and chronic. Acute GVHD typically occurs in the first 3 months after transplantation and may involve the skin, intestine, or the liver. High-dose corticosteroids such as prednisone are a standard treatment.

Chronic GVHD may also develop after haplotype matched transplant and typically occurs after the first 3 months following transplant. It is the major source of late treatment-related complications, although it less often results in death. In addition to inflammation, chronic GVHD may lead to the development of fibrosis, or scar tissue, similar to scleroderma; it may cause functional disability and require prolonged immunosuppressive therapy.

“Acute transplant rejection” is the rejection by the immune system of a transplanted organ. Acute rejection is characterized by infiltration of the transplanted tissue by immune cells of the recipient, which carry out their effector function and destroy the transplanted tissue. The onset of acute rejection is rapid and generally occurs in humans within a few weeks after transplant surgery.

Generally, acute rejection is inhibited or suppressed with immunosuppressive drugs. Steroids are the mainstay of therapy for acute rejection episodes. The typical dosage is 3-5 mg/kg/d for 3-5 days, which is then tapered to a maintenance dose. ATG and muromonab-CD3 also find use.

“Chronic transplant rejection” generally occurs in humans within several months to years after engraftment, even in the presence of successful immunosuppression of acute rejection. Fibrosis is a common factor in chronic rejection of all types of organ transplants. Chronic rejection can typically be described by a range of specific disorders that are characteristic of the particular organ. For example, in lung transplants, such disorders include fibroproliferative destruction of the airway (bronchiolitis obliterans); in heart transplants or transplants of cardiac tissue, such as valve replacements, such disorders include fibrotic atherosclerosis; in kidney transplants, such disorders include, obstructive nephropathy, nephrosclerorsis, tubulointerstitial nephropathy; and in liver transplants, such disorders include disappearing bile duct syndrome.

Chronic rejection can also be characterized by ischemic insult, denervation of the transplanted tissue, hyperlipidemia and hypertension associated with immunosuppressive drugs. Unless inadequate immunosuppression is the cause of rejection, changes in immunosuppressive therapy are generally not effective in reversing chronic rejection. Control of blood pressure, treatment of hyperlipidemia, and management of diabetes are the current mainstays of treatment for graft preservation.

The term “transplant rejection” encompasses both acute and chronic transplant rejection. In transplant rejection, the transplanted tissue is rejected and destroyed by the recipient's immune system. Acute rejection may occur to some degree in all transplants, except in the cases of identical twins or during immunosuppression. Acute rejection may begin as soon as one week after transplant and greatest risk for development of acute rejection occurs in the first three months following transplant. Chronic rejection is the long-term loss of function of a transplanted organ.

Hematopoietic cell transplant loss is the absence of hematopoietic reconstitution of donor origin on day +45 after the allograft (primary graft rejection) or as confirmed loss of donor cells after transient engraftment of donor-origin hematopoiesis. Kidney graft failure is creatinine clearance declining to less than 10 ml/min or the return of the patient to dialysis, or the return of the patient to the transplant list for re-transplantation.

Chimerism, as used herein, generally refers to chimerism of the hematopoietic system, unless otherwise noted. A determination of whether an individual is a full chimera, mixed chimera, or non-chimeric made be made by an analysis of a hematopoietic cell sample from the graft recipient, e.g. peripheral blood, bone marrow, etc. as known in the art. Analysis may be done by any convenient method of typing. In some embodiments the degree of chimerism amongst all mononuclear cells, T cells, B cells, CD56+ NK cells, and CD15+ neutrophils is regularly monitored, using PCR with probes for microsatellite analysis. For example, commercial kits that distinguish polymorphisms in short terminal repeat lengths of donor and host origin are available. Automated readers provide the percentage of donor type cells based on standard curves from artificial donor and host cell mixtures.

Individuals who exhibited more than a 95% donor cells in a given blood cell lineage by such analysis at any time post-transplantation are referred to as having full donor chimerism in this transplant patient group.

“Diagnosis” as used herein generally includes determination of a subject's susceptibility to a disease or disorder, determination as to whether a subject is presently affected by a disease or disorder, prognosis of a subject affected by a disease or disorder (e.g., identification of pre-metastatic or metastatic cancerous states, stages of cancer, or responsiveness of cancer to therapy), and use of theranostics (e.g., monitoring a subject's condition to provide information as to the effect or efficacy of therapy).

The term “biological sample” encompasses a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease.

“Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The terms “individual,” “subject,” “host,” and “patient,” used interchangeably herein and refer to any mammalian subject for whom diagnosis, treatment, or therapy is desired, particularly humans.

The term “graft management” refers to therapeutic methods that induce and/or promote repair engraftment of a solid organ, but not limited to, kidney transplantation.

The term “pharmaceutically acceptable” as used herein refers to a compound or combination of compounds that will not impair the physiology of the recipient human or animal to the extent that the viability of the recipient is compromised. Preferably, the administered compound or combination of compounds will elicit, at most, a temporary detrimental effect on the health of the recipient human or animal.

The term “carrier” as used herein refers to any pharmaceutically acceptable solvent of agents that will allow a therapeutic composition to be administered directly to a wound of the skin. The carrier will allow a composition to be topically applied to an exposed surface of an organ for transplantation and the site of the recipient where the organ is to be placed. A “carrier” as used herein, therefore, refers to such solvent as, but not limited to, water, saline, oil-water emulsions, or any other solvent or combination of solvents and compounds known to one of skill in the art that is pharmaceutically and physiologically acceptable to the recipient human or animal.

The term “assessing” and “evaluating” are used interchangeably to refer to any form of measurement, and includes determining if an element is present or not. The terms “determining,” “measuring,” “assessing,” and “assaying” are used interchangeably and include both quantitative and qualitative determinations. Assessing may be relative or absolute. “Assessing the presence of” includes determining the amount of something present, as well as determining whether it is present or absent.

### Methods of Use and Cell Compositions

Aspects of the present disclosure include methods and composition that provide organ and tissue transplant tolerance to recipients of living related and unrelated, and deceased donor organs (kidney, heart, lungs, liver and bowel), and tissue and composite tissue transplants that include all degrees of HLA mismatch. An aspect of the present disclosure provides a TLI-ATG recipient conditioning regimen. An aspect of the present disclosure provides a composition of matter for the donor cell inoculum. The regimen and composition when combined together will be expected to establish persistent mixed donor cell chimerism in recipients of living related and unrelated, and deceased donor organ transplants of all degrees of HLA mismatch. Persistent mixed chimerism will support IS drug minimization and/or complete IS drug cessation while preventing rejection of the organ grafts. IS drug minimization (defined as maintenance low therapeutic dose single agent IS monotherapy) is not expected to be associated with the medical co-morbidities caused by the current multi-IS drug regimens.

Aspects of the disclosures herein include methods and compositions that are administered to patients with relapsing and refectory autoimmune disorders. There is abundant evidence for a critical role of the immune system in pathogenesis of these diseases and T-, B-, Natural Killer (NK), and Regulatory T (Treg) cells are involved. Targeting these immune cell types with a number of therapeutic monoclonal antibodies, cytokine blockers, and integrin blockers can successfully provide disease control and relief of patient symptoms. For example, natalizumab blocks entry of lymphocytes into the CNS by binding to α4β1 integrins, infliximab blocks TNF-alpha, alemtuzumab depletes T and B cells by binding to CD52, and both ocrelizumab and rituximab deplete B cells by binding to CD20. All classes of biologic disease modifying therapies for the treatment of autoimmune disorders display efficacy yet none “re-set” and “re-store” the immune dysregulation that underlie the disease pathogenesis. Similar to organ and tissue transplantation, long-term and continued maintenance therapy is required. With continued use all classes of biologic disease modifying drugs are associated with a risk of developing significant medical co-morbidities (serious infections including tuberculosis, bacterial infections, including sepsis and pneumonia, invasive fungal, viral and other opportunistic infections, progressive multi-focal leukoencephalopathy, lymphoma, cancers, hepatobiliary diseases, congestive heart failure and autoimmune-like disorders). The administration of recipient conditioning using TLI-svldTBI combined with infusion of hematopoietic cell subsets may ‘reset’ and ‘restore’ the immune dysregulation underlying the autoimmune disease, and provide immune tolerance in a manner that will enable highly efficacious and durable disease control.

Aspects of the present disclosure include a TLI-ATG recipient conditioning regimen that can be used in organ, and tissue transplantation and autoimmune tolerance protocols. The methods include administering a single, very low dose of TBI (svldTBI). The conditioning regimen herein called, “TLI-svldTBI-ATG” is described below (at times and in some cases ATG may be omitted). The use of a svldTBI induces a profound recipient immune cell depletion above and beyond that which is induced by TLA, ATG, or the combination of TLI and ATG, by specifically targeting non-lymphoid-tissue resident immune cells that are not targeted by TLI, ATG, or TLI-ATG. Consequently TLI-svldTBI⁺/₋ATG results in an outcome significantly different than TLI-ATG. As a result of the enhanced depletion of tissue resident host immune cells that mediate resistance to donor hematopoietic cell engraftment, far fewer numbers of donor CD34+ hematopoietic stem cells and their subsets can be used to promote donor hematopoietic cell engraftment and persistent mixed chimerism. The svldTBI is too low a dose of TBI to create “marrow space”, and too low a dose to induce the toxicities associated with TBI-based recipient regimens used in BMT protocols that result in conversion to complete donor type hematopoiesis such as marrow hypoplasia with severe cytopenias, mucositis, and other GI toxicities. Targeting non-lymphoid resident tissue recipient immune cells without inducing marrow hypoplasia results in improved rates of persistent mixed donor hematopoietic cell chimerism and avoid the risks of graft-versus-host disease (GVHD).

A modified improvement to TLI-ATG host conditioning enhances persistent mixed donor cell chimerism when a single TLI dose is replaced with a single, very low dose of TBI. The TBI dose is far lower than any previously used in allogeneic hematopoietic cell transplantation regimens. The single, very low dose of TBI is used in a novel manner to de-bulk tissue resident memory T cells residing outside the fields of TLI rather than to induce marrow hypoplasia. Eradicating host tissue resident memory T cells facilitates persistent donor cell chimerism following combined organ and same donor hematopoietic cell transplantation from living related, and unrelated donors of all degrees of HLA mismatch or from deceased donors. The modified and improved host conditioning of TLI-svldTBI-ATG is designed to be combined with a novel and non-physiologic ratio of donor blood or marrow (or spleen) derived CD34+ and CD3+ T cells that constitute a unique ‘composition of matter’.

The subject methods can combine the use of TLI-ATG conditioning with a single very low dose of TBI to promote persistent mixed hematopoietic cell chimerism following the infusion of donor hematopoietic cells from HLA mismatched living or deceased organ donors.

TLI-ATG is administered in the regular manner, yet one dose of TLI is omitted, and instead a single, very low dose of TBI (svldTBI), 40-140 cGy is administered. A single TBI dose of less than 200 cGy has not previously been administered to humans, in part, because a single dose less than 200 cGy is not expected to induce enough marrow hypoplasia to facilitate donor cell engraftment and chimerism. The svldTBI (40-140 cGy) is also not expected to induce marrow hypoplasia, rather the svldTBI provides enhanced host lympho-depletion and without increasing recipient organ toxicity owning to the single very low dose. Unlike TLI, TBI does not shield the gut, liver and lungs, and consequently the large immune cell reservoirs residing within these organs will be partially depleted following the single, very low dose of irradiation. The enhanced non-lymphoid immune cell depletion removes resistance to donor cell engraftment, and allows persistent mixed chimerism following infusions of hematopoietic cells from living related and unrelated donors with all degrees of HLA mismatch, and from deceased donors. The TLI-svldTBI-ATG regimen can protect against GVHD as mixed chimerism is protective.

The dose of TLI is otherwise conventional, providing for a total dose of about 8 Gy, usually a total dose of about 7.2 Gy to account for the svldTBI, fractionated in doses of 0.8 Gy, with about 2 fractions/week.

In some cases, ATG is included, e.g. delivered intravenously. In some cases, a single dose of ATG may be delivered to the recipient. In other cases, the recipient may receive more than one dose of ATG. For example, a recipient may receive at least one dose of ATG, two doses of ATG, three doses of ATG, four doses of ATG, five doses of ATG, six doses of ATG, seven doses of ATG, eight doses of ATG, nine doses of ATG, 10 doses of ATG, 11 doses of ATG, 12 doses of ATG, 13 doses of ATG, 14 doses of ATG, 15 doses of ATG, 16 doses of ATG, 17 doses of ATG, 18 doses of ATG, 19 doses of ATG, or at least 20 doses of ATG. In some cases, each dose of ATG is at least about 0.1 mg/kg, at least about 1 mg/kg, at least about 5 mg/kg, up to about 20 mg/kg. In some cases, the ATG is delivered intra-operatively before the transplanted organ is perfused with host blood. In other cases, the ATG is delivered intra-operatively after the transplanted organ is perfused with host blood. In some cases, the ATG is delivered intra-venously before the transplanted organ is perfused with host blood. In other cases, the ATG is delivered intra-venously after the transplanted organ is perfused with host blood. In some cases, the ATG is delivered intra-arterially before the transplanted organ is perfused with host blood. In other cases, the ATG is delivered intra-arterially after the transplanted organ is perfused with host blood. In some cases, the ATG is delivered subcutaneously before the transplanted organ is perfused with host blood. In other cases, the ATG is delivered subcutaneously after the transplanted organ is perfused with host blood. In some cases, the ATG is delivered intraperitoneally before the transplanted organ is perfused with host blood. In other cases, the ATG is delivered intraperitonially after the transplanted organ is perfused with host blood.

Corticosteroid therapy may be given as medication prior to administration of ATG. In some cases, solumedrol may be administered although any corticosteroid known to one of skill in the art sufficient to reduce side effects of ATG may be used at an effective dose. In some cases, the corticosterioid may be administered on the same day as ATG is administered.

Following the final dose of ATG administered to the recipient, prednisone may be administered. In some cases, a single dose of prednisone may be administered. In other cases, more than one dose of prednisone may be administered. For example, multiple doses of prednisone may be administered according to a tapering course or a constant course.

### Typing Human Leukocyte Antigens

In some cases, the methods described herein may comprise the steps of: HLA typing a donor and recipient to determine an HLA-matched or HLA-mismatched pair. “HLA-matched” indicates all of the 8 high expression HLA antigens (e.g., HLA-A, B, DR) are matched between a donor and a recipient. “HLA-mismatched” indicates that at least 1, at least 2, at least 3 or more of 8 HLA antigens (e.g., HLA-A, B, C, DR) are mismatched. Generally at least a portion of the 8 HLA antigens (e.g., HLA-A, B, C, DR) are matched, for example at least 1, at least 2, at least 3, at least 4, at least 6 matches.

In some cases, the methods may include at least the following steps; obtaining the solid organ and hematopoietic cells from the donor; isolating hematopoietic cells of the appropriate type and dose; transplanting the solid organ; performing a conditioning regimen on the recipient following transplantation of the solid organ and prior to infusion of the engineered hematopoietic cells; maintaining the recipient on an immunosuppressive regimen for at least six months; monitoring the recipient for mixed chimerism of the hematopoietic system; and withdrawing immunosuppression if the recipient shows stable mixed chimerism. The methods described herein apply to both HLA-matched and HLA-mismatched transplantation conditions.

Individuals selected for the methods described herein may meet the criteria of (i) requiring a solid organ graft; and (ii) having either an HLA-matched or HLA-mismatched donor from which the solid organ and hematopoietic cells can be obtained. By performing a combined transplant of solid organ and an engineered hematopoietic cell infusion appropriate for the individual, in combination with non-myeloablative conditioning, the patient may have a high probability of developing persistent mixed chimerism for at least 6 months. Mixed chimerism which persists for at least 6 months may allow for withdrawal of immunosuppression over time.

Any method known in the art may be used to type donor-derived cells and a sample from the recipient. For example, three main procedures may be used to perform HLA typing. The first is conventional serological cytotoxicity method, where samples of lymphocytes (e.g., taken from blood or spleen) are added to Terasaki plates. In some cases, B lymphocytes may be used for class II typing. In other cases, class I typing may be performed with the remaining leucocytes. Magnetic beads may be used to purify cells from blood or spleen.

In some cases, each of the wells of the Terasaki plates may contain a plurality of antibodies (e.g., from either maternal sera or manufactured monoclonal antibodies). In some cases, the HLA antigen expressed by a cell binds to an antibody in the well. After the addition of complement, cells located in a well where the HLA antigen and antibody were bound may be killed. In some cases, a pattern of cell death may be determined from the wells. The pattern may allow for deduction of the combination of HLA antigens that were present on the original tissue. In some cases, the deduction of the combination of HLA antigens may result in typing of HLA antigens.

Another method that may be used for HLA typing is flow cytometry. Unlike the conventional serological cytotoxicity method, flow cytometry may be used to identify one or more HLA alleles. In this method, leukocytes may be combined with antibodies that bind to the HLA types of interest. In some cases the antibodies may be monoclonal or polyclonal. In some cases, the antibodies may contain a detectable label. In some cases, the antibodies may be directly conjugated to a detectable label. In other cases, a different antibody with a detectable label binds to the HLA antibody and the complex is then detected. The types of detectable labels that may be used for HLA typing by flow cytometry are readily available and known to those of skill in the art. The sample may be analyzed to determine which HLA antibodies have bound to the cells.

Yet another method that may be used for HLA typing is DNA typing. In some cases, DNA typing involves extracting DNA from cells and amplifying the genes that encode for the HLA peptides using polymerase chain reaction techniques which generate sequence data. The polymerase chain reaction techniques may include any polymerase chain reaction technique which generates sequence data that is known to one of skill in the art.

In some cases, the sequence of the genes may be matched with the known nucleotide sequences of HLA alleles located in at least one of several genetic (e.g., gene bank) databases. In some cases, the gene bank data base may be the IMGT/HLA (International Immunogenetics Project) database.

### Solid Organ Transplant

Solid organs may be transplanted from a donor to a recipient such that the organ is placed into the appropriate position in the recipient body. In some cases, the cardiovascular connections between the solid organ may be physiologically integrated into the recipient body. In some cases, the organ may be from a living donor. In other cases, the organ may be from a deceased donor. In some cases, the solid organ may be HLA-matched between the donor and the recipient. In other cases, the solid organ may be HLA-mismatched between the donor and the recipient.

Any solid organ that may be used for organ transplantation may be used with the methods described herein. In some cases, the organ may be a kidney, lung, pancreas, pancreatic islet cells, heart, intestine, colon, liver, skin, muscle, gum, eye, tooth and the like as known to those of skill in the art. In some cases, the organ may be a complete organ. In other cases, the organ may be a portion of an organ. In other cases, the organ may be cells from a tissue of an organ.

Using the methods described herein, the solid organ is harvested and transplanted in accordance with conventional practice. In some cases, the solid organ may be transplanted at least one, two, three, four, five, six, seven, eight, nine, ten, eleven, twelve, thirteen, fourteen, fifteen, sixteen, seventeen, eighteen, nineteen or at least twenty days prior to the infusion of the engineered hematopoietic cells.

### Obtaining Hematopoietic Stem Cells for Transplantation

Hematopoietic stem cell transplantation (HCT) includes the transplantation of multipotent hematopoietic stem cells from a donor to a recipient. For the methods described herein, HCT may be combined with solid organ transplant. In some cases, the hematopoietic stem cells may be HLA-matched between the donor and the recipient. In other cases, the hematopoietic stem cells may be HLA-mismatched between the donor and the recipient.

In some cases, the hematopoietic stem cells are isolated and purified from the solid organ donor. The solid organ donor may be living or deceased. In cases of a living donor, hematopoietic cells may be obtained from the solid organ donor using any of the various methods known to one of skill in the art, including apheresis of mobilized peripheral blood from living donors; harvesting hematopoietic cells from bone marrow of deceased donors, and the like. In cases of a deceased donor, hematopoietic cells may be obtained from bone marrow. For example, in a deceased donor the cells may be obtained from the spleen, from bone marrow in vertebrae, pelvic bone, femur or any other bone which contains sufficient bone marrow from which to extract hematopoietic cells.

In some cases, hematopoietic cells may be mobilized prior to isolation and purification. In some cases hematopoietic cells may be mobilized by treating the donor with granulocyte colony stimulating factor (G-CSF). For example, the donor may be treated with one, two, three, four, five, six, seven, eight, nine, ten or more than ten doses of G-CSF prior to isolating and purifying hematopoietic cells. In some cases, the doses of G-CSF may be delivered to the donor on a single day (e.g., a 24 hour day) or over the course of multiple days. For example, multiple days may include two, three, four, five, six, seven, eight, nine, ten or more than ten days. In a preferred case, the donor receives two doses per day. In some cases, each dose of G-CSF delivered to the donor is a conventional dose, e.g. from about 1 to about 20 micrograms/kg of donor body weight. In other cases, each dose of G-CSF delivered to the donor is about 8 micrograms/kg of donor body weight. In some cases, apheresis may be performed after the donor receives a single dose of G-CSF. For example, apheresis may be performed from about one hour to about 48 hours, or more than 48 hours after the donor receives the single dose of G-CSF. In other cases, apheresis may be performed after the donor receives the final dose of multiple doses of G-CSF. One or more apheresis products may be obtained from a donor.

In some embodiments, the hematopoietic cells are obtained from a solid organ donor HLA-matched to the recipient. In this case, the hematopoietic cells are HLA-matched to the solid organ and the solid organ recipient. In other cases, the hematopoietic cells may be obtained from a solid organ donor HLA-mismatched to the recipient. In this case, the hematopoietic cells are HLA-matched to the solid organ and HLA-mismatched to the solid organ recipient.

For the methods described herein, hematopoietic cells may be frozen (e.g., cryopreserved) after isolation or after isolation and purification from the solid organ donor. In some cases, hematopoietic cells may be cryopreserved using a cryopreservation medium and a method of cryopreservation known to those of skill in the art. In some cases, the hematopoietic cells may be cryopreserved using a cryopreservation medium containing dimethylsulfoxide (DMSO), Normosol, Hetastarch and human serum albumin (HSA). In some cases, the cryopreservation medium may contain other components in order to cryopreserve the hematopoietic cells in accordance with and for use with the methods described herein.

For the methods described herein, hematopoietic cells can be frozen (e.g., cryopreserved) after isolation or after isolation and purification from the solid organ donor. In some cases, hematopoietic cells may be cryopreserved using a cryopreservation medium and method of cryopreservation known to those of skill in the art. In some cases, the hematopoietic cells may be cryopreserved using a cryopreservation medium containing dimethylsulfoxide (DMSO), fetal calf serum (FCS) and RPMI medium. In some cases, the cryopreservation medium may contain other components in order to cryopreserve the hematopoietic cells in accordance with and for use with the methods described herein.

Cryopreservation of hematopoietic cells includes a process of controlled rate freezing the cells once contained within cryopreservation medium. In some cases, a cryofreezer equipped with a computer to control the rate and temperatures of controlled rate freezing can be used to perform cryopreservation of the hematopoietic cells. For example, the hematopoietic cells may be placed in a cryofreezer with a chamber temperature at or below 6.5° C. The computer may control the rate and temperatures of controlled rate freezing such that the cryofreezer reaches a temperature of at least or below −130° C. such that the hematopoietic cells are preserved in manner in accordance with the methods described herein. In some cases, the cryofreezer uses liquid nitrogen to control the temperature of the freezer at which the hematopoietic cells are stored. In some cases, the hematopoietic cells may be cryopreserved and stored in a cryofreezer prior to delivery to the recipient. In some cases, the hematopoietic cells may be cryopreserved for less than from about one month to less than about 60 months. In some cases, the hematopoietic cells may be cryopreserved for less than one year to less than about 60 years. In some cases, cryopreservation may result in hematopoietic cell death which is determined upon thawing of the cells prior to infusion into the recipient. Using conventional methods of determining cell death (e.g., trypan blue staining, flow cytometry, etc.) known to those of skill in the art, the percent of dead cells in batch of cryopreserved hematopoietic cells may be determined.

### Isolation and Purification of Donor Hematopoietic Stem Cells and Unique Immune Cell Subsets

For the methods described herein, hematopoietic stem cells may be derived from bone marrow, peripheral blood (by apheresis of blood mononuclear cells), umbilical cord blood, spleen, lymph nodes, etc. In some cases, the hematopoietic stem cells and immune cells may be HLA-matched between the donor and the recipient. In other cases, the hematopoietic stem cells and immune cells may be HLA-mismatched between the donor and the recipient.

In some cases, specific subsets of cells and unique populations of cells are isolated and purified from the source of hematopoietic cells. In some cases, the cells that are isolated and purified are CD34⁺ cells; and immune cell subsets are isolated from CD3⁺ cells. In some cases, the CD34⁺ and CD3⁺ cells are isolated from the same fraction of hematopoietic cells. In some cases, the CD34⁺ and CD3⁺ cells are isolated from a different fraction of hematopoietic cells. In some cases, the CD34⁺ cells are progenitor cells. In some cases, the CD3⁺ cells are T cells.

In some cases, CD34⁺ cells are isolated and purified from the donor hematopoietic cells. For example, CD34⁺ cells may be isolated and purified from the donor hematopoietic cells by selectively binding a suitable CD34 affinity reagent. In some cases, a CD34 affinity reagent may be an antibody, a full-length antibody, a fragment of an antibody, a naturally occurring antibody, a synthetic antibody, an engineered antibody, a full-length affibody, or a fragment of any of the above. In some cases, the affinity reagent is directly conjugated to a detection reagent and/or purification reagent. In some cases, the detection reagent and purification reagent are the same. In other cases, the detection reagent and purification reagent are different. For example, the detection reagent and/or purification reagent is fluorescent, magnetic, or the like. In some cases, the detection reagent and/or purification reagent is a magnetic particle for column purification. For example, magnetic column purification may be performed using the Miltenyi system of columns, antibodies, buffers, preparation materials and reagents, etc. known to those of skill in the art. In some cases, CD34⁺ cells isolated and purified using a magnetic particle may contain iron. The iron content of isolated and purified CD34⁺ cells may be greater after isolation and purification using magnetic particles than the iron content in the CD34⁺ cells prior to isolation and purification.

In some cases, CD3⁺ cells are isolated and purified from the donor hematopoietic cells. For example, CD3⁺ cells may be isolated and purified from the donor hematopoietic cells by selectively binding a suitable CD3 affinity reagent. In some cases, a CD3 affinity reagent may be an antibody, a full-length antibody, a fragment of an antibody, a naturally occurring antibody, a synthetic antibody, an engineered antibody, a full-length affibody, a fragment of an affibody, a full-length affilin, a fragment of an affilin, a full-length anticalin, a fragment of an anticalin, a full-length avimer, a fragment of an avimer, a full-length DARPin, a fragment of a DARPin, a full-length fynomer, a fragment of a fynomer, a full-length kunitz domain peptide, a fragment of a kunitz domain peptide, a full-length monobody, a fragment of a monobody, a peptide, a polyaminoacid, or the like.

In some cases, the affinity reagent is directly conjugated to a detection reagent and/or purification reagent. In some cases, the detection reagent and purification reagent are the same. In other cases, the detection reagent and purification reagent are different. For example, the detection reagent and/or purification reagent is fluorescent, magnetic, or the like. In some cases, the detection reagent and/or purification reagent is a magnetic particle for column purification. For example, magnetic column purification may be performed using the Miltenyi system of columns, antibodies, buffers, preparation materials and reagents, etc. known to those of skill in the art.

In some cases, both of the CD34+ and CD3+ cells isolated and purified using a magnetic particle may contain iron. The iron content of isolated and purified CD34+ and CD3+ cells may be greater after isolation and purification using magnetic particles than the iron content in the CD34+ and CD3+ cells prior to isolation and purification.

### Engineering and Preparing Hematopoietic Stem Cell and Immune Cells for Pharmaceutical Compositions

A combination of CD34+ and CD3+ cells derived from the donor using the methods described herein may be engineered into a pharmaceutical composition for administration to the solid organ recipient. In some cases, the hematopoietic cells may be engineered into a single pharmaceutical composition for infusion into a recipient. In other cases, the hematopoietic cells may be engineered into multiple pharmaceutical compositions for infusion into a recipient. In some cases, the CD34+ and CD3+ cells may be HLA-matched between the donor and the recipient. In other cases, the CD34+ and CD3+ cells may be HLA-mismatched between the donor and the recipient.

In some cases, the hematopoietic cells may be engineered into a pharmaceutical composition having a pre-determined purity of CD34+ hematopoietic cells prior to mixing with additional cells is at least ≥50% purity, ≥55% purity, ≥60% purity, ≥65% purity, ≥70% purity, ≥75% purity, ≥80% purity, ≥85% purity, ≥90% purity, ≥95% purity or ≥98% purity. In an exemplary case, the purity of the CD34+ progenitor cells in the engineered hematopoietic cells is ≥70% purity.

For example, the purity of the CD3⁺ cells in the engineered hematopoietic cells may be ≥30% purity, ≥40% purity, ≥50% purity, ≥55% purity, ≥60% purity, ≥65% purity, ≥70% purity, ≥75% purity, ≥80% purity, ≥85% purity, ≥90% purity, ≥95% purity or ≥98% purity. In another example, the purity of the CD3+ cells in the engineered hematopoietic cells may be between 10 and 30% purity, 15 and 35% purity, 20 and 40% purity, 25 and 45% purity, 30 and 50% purity, 35 and 55% purity, 40 and 60% purity, 45 and 65% purity, 50 and 70% purity, 55 and 75% purity, 60 and 80% purity, 65 and 85% purity, 70 and 90% purity, 75 and 95% purity, and 80 and 100% purity. In an exemplary case, the purity of the CD3+ cells in the engineered hematopoietic cells is ≥70% purity prior to combining with the CD34+ cells.

In the case of living HLA mismatched related and unrelated donors: donor hematopoietic cells can be mobilized using granulocyte colony stimulating factor (G-CSF)+/−mozobil, and the donor will undergo 1 or 2 consecutive days of high volume (>12 liters) blood apheresis to obtain blood mononuclear cells. The apheresis collection(s) will be processed for CD34⁺ cell enrichment using either fluorescence activated cell sorting (FACS) or magnetic activated cell sorting (MACS) as per manufacturer's guidelines.

The CD34⁺ enriched product is cryopreserved in the standard manner. The pre-freeze CD34⁺ cell purity is at least about ≥70%. The CD34+ cell dose will have a pre-freeze value of from about 4 to about 20×10⁶ CD34+ cells/kg recipient weight, for example from about 4×10⁶ CD34⁺ cells/kg; from about 10×10⁶ CD34⁺ cells/kg, from about 12×10⁶ CD34⁺ cells/kg, from about 14×10⁶ CD34⁺ cells/kg, from about 16×10⁶ CD34⁺ cells/kg, from about 18×10⁶ CD34⁺ cells/kg, from about 20×10⁶ CD34⁺ cells/kg.

In some cases, the non-CD34⁺ cell fraction following the CD34⁺ enrichment step is used to obtain a defined dose of CD3⁺ T cells, and will be cryopreserved in the usual manner. The pre-freeze dose of CD3⁺ cells is from about 25 to about 100×10⁶ CD3⁺/kg recipient weight, for example from about 25×10⁶ CD3⁺/kg, from about 35×10⁶ CD3⁺/kg, from about 45×10⁶ CD3⁺/kg, from about 50×10⁶ CD3⁺/kg, up to about 100×10⁶ CD3⁺/kg, up to about 90×10⁶ CD3⁺/kg, up to about 80×10⁶ CD3⁺/kg, up to about 70×10⁶ CD3⁺/kg, up to about 60×10⁶ CD3⁺/kg.

In some embodiments, enriched populations of donor derived CD8⁺ memory T cells, which can be defined as CD3⁺/CD8⁺/CD45RA⁻/CD45RO⁺ are provided at a dose of from about 1 to about 12×10⁶ cells/kg, for example from about 1×10⁶ cells/kg, from about 2×10⁶ cells/kg from about 4×10⁶ cells/kg, from about 6×10⁶ cells/kg, to about 12×10⁶ cells/kg, to about 10×10⁶ cells/kg, to about 8×10⁶ cells/kg. The memory cells may be infused from about 0 to about 3 days after the CD34⁺ enriched cell product, for example from about 0 to 3, from about 1-3, from about 2-3 days following the CD34⁺ enriched cell product. In some embodiments the CD8+ memory T cells are provided in the place of CD3+ cells.

In some embodiments, donor derived Treg cells, which can be defined as CD4⁺CD25⁺FoxP3⁺ enriched by FACS or MACS methods are infused from about 0 to about 4 days after the infusion of donor CD34⁺ enriched cells; at a dose of from about 1 to about 10×10⁶ cells/kg, for example from about 1×10⁶ cells/kg, from about 2×10⁶ cells/kg from about 4×10⁶ cells/kg, from about 6×10⁶ cells/kg, to about 12×10⁶ cells/kg, to about 10×10⁶ cells/kg, to about 8×10⁶ cells/kg. The Treg cells may be infused from about 0 to about 4 days after the CD34⁺ enriched cell product, for example from about 0 to 3, from about 1-3, from about 2-3 days following the CD34⁺ enriched cell product. In some embodiments, the donor Treg cells are combined with donor CD3+ T cells at a ratio of Treg:CD3+ T cells ranging from 1:50; 1:20, 1:10, 1:5, 1:2, 2:1, to 3:1. In some embodiments, the donor Treg cells are combined with donor CD8⁺ memory T cells at a ratio of Treg:CD8⁺ memory T cells ranging from 1:20, 1:10, 1:5, 1:3, 1:2, to 1:1.

In some cases, a manipulated cellular composition comprises a CD34⁺ cell to CD3⁺ T cell ratio of about 1:1 to about 1:15, for example of about 1:1, about 1:2, about 1:4, about 1:6, about 1:8, about 1:10, about 1:12, and not more than about 1:15. If the absolute number of CD34⁺ cells is consistently less than the lower limit of 25 million/kg recipient weight needed to establish persistent mixed chimerism, then deceased donor splenocytes can be used to obtain additional CD34⁺ cells that will be added to the ddVB-BMCs. If the absolute number of CD3+ T cells is consistently less than the lower limit of 40 million/kg recipient weight needed to establish persistent mixed chimerism, then deceased donor splenocytes can be used to obtain and augment the CD3⁺ T cell dose to fulfill the desired threshold of 25-100 million/kg.

Aspects of the present disclosure include a composition of the donor hematopoietic cells (HC) or bone marrow cells (BMC) infused after TLI-svldTBI-ATG conditioning that will support persistent mixed chimerism. Hematopoietic cells obtained from HLA mismatched living donors are referred to as mmLD-HC, and bone marrow cells obtained from deceased donor vertebral bodies are ddVB-BMC. In both instances, mmLD-HC and ddVB-BMC, the cell composition represents a unique combination and pairing of (a unique ratio of) CD34+ cell populations with CD3+ T cells that is not intuitively obvious. The compositions can include the ratios of CD34+ cells to CD3+ T cells that will result in persistent mixed chimerism when combined with host TLI-svldTBI-ATG conditioning.

In some cases, for G-mobilized grafts from mmLD-HC when combined with TLI-svldTBI-AIG host conditioning the CD34:CD3 cell ratio is not intuitive or physiologically occurring and will approximate from 1:2 to 1:10 compared to the physiologic ratio of about 1:50 for traditional unmanipulated G-mobilized grafts.

In some cases, for ddVB-BMC the CD34⁺:CD3⁺ cell ratio when combined with TLI-svldTBI-AIG host conditioning will approximate 1:2 to 1:5, compared to a 1:10 to as high as 1:20 ratio as has been described for the use of an unmanipulated bone marrow harvest used for over four decades in clinical BMT for cancer patients.

Isolated and purified CD34+ cells and CD3+ cells may be freshly isolated or frozen (e.g., cryopreserved) prior to use in an engineered hematopoietic cell composition. In some cases, the CD34+ cells may be combined with the CD3+ cells prior to use as freshly isolated or frozen cells for preparing an engineered hematopoietic cell composition.

In the cases of deceased donor organs, donor hematopoietic and immune cells are obtained from vertebral bodies (VBs), pelvic bones, and spleen and cryopreserved in the usual manner. The deceased donor hematopoietic and immune cells will be thawed and infused into the recipient following host TLI-svldTBI-ATG conditioning. This is the first in-human application of a host conditioning regimen combined with a uniquely defined hematopoietic and immune cell product to establish persistent mixed donor cell chimerism using cells obtained from deceased donors. Persistent mixed chimerism will lead to organ transplantation tolerance, and IS drug minimization and/or withdrawal.

To obtain deceased donor bone marrow cells from the VBs we transect the VB at the vertebral arch and in a unique procedural step apply a razor thin high-pressure saline jet stream to “power-wash” away connective tissue and necrotic surgical/bacterial/cellular debris from the VB. After the VB is power-washed, a rotary saw slices open the VBs and pea sized chunks are subsequently made. Taken together these methods maximize the VB bone marrow surface area that allows maximum cell extraction and yield. The cell product is passed through a multi-sieve elution and purification step. These novel methods significantly improve VB cell yields and purity compared to previously published procedures and methods.

Using VB bone marrow cells the CD34+ cell dose range will be 2-20 million/kg recipient weight and the CD3+ T cell dose range will be 10-100 million/kg.

In some embodiments splenocytes supplement the VB bone marrow cell inoculum. Several (typically 2-8) 2 inch-sized splenic cubes removed from the donor spleen are needed as a supplemental immune cell source to support persistent mixed donor cell chimerism. The splenic cubes are harvested during the time of organ procurement and transported in standard transport media along with the donor VBs. A single cell suspension consisting of live mononuclear splenocytes is obtained by dissociating the cells from the splenic tissue using a specialized dissociation media and techniques to prevent i) over-digestion by chemical and proteolytic enzymes, and ii) excessive tissue disaggregation from environmental stress by excessive mechanical forces, vortexing, homogenization, abnormal osmolality stresses or combinations thereof. The single cell suspension will be passed through a multi-sieve elution tower with a final 80-120 micron strainer. The cell pellet is prepared for cryopreservation with or without MACS/FACS separation of the live cells for aliquots of CD3+ cells, and Treg cells, mesenchymal stem cells (MSCs), B cells, invariant natural killer (iNK) cells and hematopoietic cell precursors. These cell types can be used in cell expansion protocols which may allow for the treatment of one or more recipients.

In some cases the splenic CD3+ T cells will be added to the infused VB bone marrow cells to augment the donor CD3 T cell dose if it is low (for example in the case of using deceased donor cells and if less than 50 million CD3+ T cells are obtained from the VB bone marrow cells). In some cases, the splenic T cells will be added to enable CD3+ T cells doses that may be as high as 200 million CD3+ T cells/kg. In some cases the splenocytes will be used to exclusively obtain Treg cells to be used in doses of 1-10 million/kg recipient weight. In some cases the splenic Treg cells may be engineered with a predetermined antigen-specificity via transfection of viral vectors encoding specific T cell receptors (TCRs) or chimeric antigen receptors (CARs). The engineered Treg cells may express tissue specific antigens that promote Treg cells trafficking, migrating and residing in selected recipient tissues (bone marrow, lymph nodes, neuronal, heart, lung, kidney, liver, bowel, and pancreas) to promote local immune suppressive reactions that enhance persistent mixed chimerism and/or tissue-specific tolerance. Treg may be used as primary cells or in culture expansion and potentially in multiple recipients. In some cases, a “left over” fraction of the VB bone marrow and/or splenocytes is cryopreserved and stored for months to years, and can be given as a later donor cell boost if chimerism and/or tolerance is waning over time.

In some cases, the CD34+ and CD3+ cells are maintained independently either as freshly isolated cells or as cryopreserved cells. For example, CD34+ cells and CD3+ cells freshly maintained may be combined such that the target doses of CD34+ and CD3+ cells are achieved in the engineered composition for infusion. In other cases, CD34+ and CD3+ cells cryopreserved independently may be thawed and the target doses of each cell type determined after thawing. The thawed CD34+ and CD3+ cells may be combined such that the target doses of CD34+ and CD3+ cells are achieved in the engineered composition for infusion.

Processing Engineered Hematopoietic Cells for Pharmaceutical Compositions

Engineered hematopoietic cells (e.g., CD34+ and CD3+ cells) may be freshly prepared or previously frozen (e.g., cryopreserved) prior to generating a pharmaceutical composition for administration to a recipient. In some cases, the CD34+ and CD3+ cells may be HLA-matched between the donor and the recipient. In other cases, the CD34⁺ and CD3⁺ cells may be HLA-mismatched between the donor and the recipient.

Methods of cryopreservation are described elsewhere herein. In some cases, one aliquot of CD34+ cells is thawed. In other cases, more than one aliquot of CD34+ cells is thawed. In some cases, one aliquot of CD3+ cells is thawed. In other cases, more than one aliquot of CD3+ cells is thawed. In some cases, one aliquot of the combination of CD34+ cells and CD3+ cells is thawed. In other cases, more than one aliquot of the combination of CD34+ cells and CD3+ cells is thawed.

In some cases, freshly prepared engineered hematopoietic cells may be expanded ex vivo using methods known to those of skill in the art. In other cases, previously frozen engineered hematopoietic cells may be expanded ex vivo using methods known to those of skill in the art. In some cases, either freshly prepared or previously frozen engineered hematopoietic cells may be expanded ex vivo by use of at least one growth factor. In some cases, more than one growth factor may be used to expand the cells. For example, a growth factor may be activin A, ADAM-10, Angiogenin, Angiopoietin-1, Angiopoietin-2, Angiopoietin-3, Angiopoietin-4, BIO, Bone Morpohogenetic Protein-2, Bone Morpohogenetic Protein-3, Bone Morpohogenetic Protein-4, Bone Morpohogenetic Protein-5, Bone Morpohogenetic Protein-6, Bone Morpohogenetic Protein-7, Brain-derived neurotrophic factor, E-cadherin, Fc chimera, cathepsin G, ch2 inhibitor II, epidermal growth factor, eotaxin, eotaxin-2, eotaxin-3, Fas, fibroblast growth factor-4, fibroblast growth factor-5, fibroblast growth factor-6, fibroblast growth factor-8b, fibroblast growth factor-8c, fibroblast growth factor-9, fibroblast growth factor-10, fibroblast growth factor-17, fibroblast growth factor-18, fibroblast growth factor, fibroblast growth factor acidic, fibroblast growth factor basic, fibroblast growth factor basic fragment 1-24 bovine, fibroblast growth factor receptor 1a, fibroblast growth factor receptor 1b, fibroblast growth factor receptor 2a, fibroblast growth factor receptor 2b fibroblast growth factor receptor 3a, fibroblast growth factor receptor 4, flt-3, flk-2 ligand, granulocyte colony stimulating factor, granulocyte-macrophage colony stimulating factor, GROa, GROb, heparin-binding EGF-like growth factor, heregulin-al EGF domain, heregulin-b1 EGF domain, heregulin B, insulin-like growth factor-1, insulin-like growth factor-II fragment 33-40, insulin-like growth factor binding protein-2, insulin-like growth factor-1, insulin-like growth factor II, interferon a, interferon aA, interferon aA/D, interferon b, interferon g, interferon, interferon g receptor 1, interleukin-1a, interleukin-1 b, interleukin soluble receptor type II, interleukin-2, interleukin-2 soluble receptor a, interleukin-2 soluble receptor b, interleukin-2 soluble receptor g, interleukin-3, interleukin-5, interleukin-6, interleukin-6 soluble receptor, interleukin-7, interleukin-8, interleukin-11, interleukin-12, leukemia inhibitory factor, LONG EGF, LONG R2 IGF-1, LYN A, macrophage inflammatory protein-1a, macrophage inflammatory protein-1 b, macrophage inflammatory protein-1 g, matrix metalloproteinase-1, matrix metalloproteinase-2, matrix metalloproteinase-9, MIG, monocyte chemotactic protein-1, monocyte chemotactic protein-3, monocyte chemotactic protein-4, monocyte chemotactic protein-5, nerve growth factor receptor, neurotrophin-3, neurotrophin-4, noggin, notch-1, oncostatin M, oncostatin M receptor b, osteopontin, osteoprotegrin, phenylarsine oxide, platelet-derived growth factor, platelet-derived growth factor-AB, platelet-derived growth factor-BB, platelet-derived growth factor soluble receptor a, platelet derived growth factor receptor b, anti-POU5F1, oct4, RANTES, SCF soluble receptor, L-selectin, stem cell factor, stromal cell-derived factor 1a, stromal cell-derived factor 1 b, thromopoietin, Tie-1, tissue inhibitor of metalloproteinase-2, transforming growth factor-a, transforming growth factor-b1, transforming growth factor-b2, transforming growth factor-b3, transforming growth factor-b1 receptor II soluble fragment, transforming growth factor-b soluble receptor III, TrkB, vascular endothelial growth factor 120, vascular endothelial growth factor 121, vascular endothelial growth factor 164, VEGF receptor-2/Flk1/KDR and/or VEGF Receptor-3/Flt-4. The amount of each growth factor used for ex vivo expansion is known to one of skill in the art and suitable for use with the methods described herein.

In some cases, either freshly prepared or previously frozen engineered hematopoietic cells may be expanded ex vivo by use of at least one type of feeder cell. Any type of feeder cell may be used such that the feeder cells maintain viability of engineered hematopoietic cells, and promote engineered hematopoietic cell proliferation and differentiation. In some cases, at least one growth factor combined with at least one feeder cell may be used such that the feeder cells maintain viability of engineered hematopoietic cells, and promote engineered hematopoietic cell proliferation and differentiation. In some cases, feeder cells may be mitotically inactive. In some cases, more than one type of feeder cell may be used to expand the cells. In some cases, a type of feeder cell may be derived from adult mouse endothelial cells, embryonic mouse endothelial cells, adult mouse fibroblasts, embryonic mouse fibroblasts, adult human endothelial cells, embryonic human endothelial cells, adult human fibroblasts, embryonic human fibroblasts, adult non-human primate endothelial cells, embryonic non-human primate endothelial cells, adult non-human primate fibroblasts, embryonic non-human primate fibroblasts, adult bovine endothelial cells, embryonic bovine endothelial cells, adult bovine fibroblasts, embryonic bovine fibroblasts, adult porcine endothelial cells, embryonic porcine endothelial cells, adult porcine fibroblasts, embryonic porcine fibroblasts and the like.

In some cases, feeder cells may be modified. For example, the modifications may be genetic. In some cases, feeder cells may express non-native genes, repress expression of native genes or overexpress native genes. For example, feeder cells may express LacZ, GFP, RFP or the like.

### Compositions of Hematopoietic Stem Cells

The hematopoietic stem cells and compositions thereof of the methods provided herein can be supplied in the form of a pharmaceutical composition, comprising an isotonic excipient prepared under sufficiently sterile conditions for human administration. Choice of the cellular excipient and any accompanying elements of the composition is adapted in accordance with the route and device used for administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000.

In some cases, the hematopoietic stem cells may be HLA-matched between the donor and the recipient. In other cases, the hematopoietic stem cells may be HLA-mismatched between the donor and the recipient.

In some cases, the pharmaceutical composition may contain agents which enhance engraftment of the hematopoietic cells in the recipient. In other cases, the pharmaceutical composition may contain agents which do not affect engraftment of the hematopoietic cells in the recipient. In some cases, the pharmaceutical composition may contain agents which prevent a negative reaction of the recipient to the hematopoietic cells. For example, any agent as mentioned above may be a cytokine, a chemokine, a growth factor, an excipient, a carrier, an inert molecule, an antibody or a fragment thereof, a small molecule, a drug, an agonist, an antagonist, a chemical or the like. Any agent used in a pharmaceutical composition of hematopoietic cells in the recipient is physiologically acceptable.

A variety of methods may be used to deliver hematopoietic cells to the recipient and any method known to one of skill in the art may be applied to the hematopoietic cells described herein. For example, the hematopoietic cells may be delivered to the recipient by injection using a needle, catheter, central line or the like. In some cases, the hematopoietic cells may be delivered intravascularly, intravenously, intraarterially, intracranially, intraperitoneally, subcutaneously, intramuscularly, intraorbitally, or through any source which permits the hematopoietic cells to home to an appropriate site in the recipient such that the hematopoietic cells persist, regenerate and differentiate in the recipient.

The composition of engineered hematopoietic cells may also comprise or be accompanied with one or more other ingredients that facilitate the engraftment or functional mobilization of the cells. For example, ingredients may include matrix proteins that support the cells, promote adhesion of the cells, or complementary cell types (e.g., endothelial cells).

In some cases, the hematopoietic cells may home to an organ, a tissue or a cell type within the recipient. For example, an organ may the brain, thyroid, eyes, skin, lungs, pancreas, spleen, bladder, prostate, kidneys, stomach, liver, heart, adrenal glands, bronchi, large intestine, small intestine, spinal cord, bone, bone marrow, pituitary gland, salivary gland, gall bladder, larynx, lymph nodes, prostate, skeletal muscles, appendix, esophagus, parathyroid glands, trachea, urethra, ovaries, testicles, uterus, ureters, fallopian tubes, or any gland in the body. In some cases, a tissue or a cell type may be part of an organ. In some cases, a tissue or a cell type may be a derived from an organ. In some cases, a tissue or a cell type may be isolated from an organ.

In some cases, the recipient of the hematopoietic stem cells may not have received a solid organ transplant. In other cases, the recipient may have received a solid organ transplant. In some cases, the solid organ transplant recipient may be administered one dose of engineered hematopoietic stem cells. In other cases, the solid organ transplant recipient may be administered more than one dose of engineered hematopoietic stem cells. In some cases, the time elapsed between each dose of engineered hematopoietic stem cells may be the same. In other cases, the time elapsed between each dose of engineered hematopoietic stem cells may be different.

For example, the solid organ transplant recipient may be administered a first dose of engineered hematopoietic stem cells at least about 1, at least about 5, at least about 10, at least about 15, at least about 20, at least about 25, at least 30 or more days following the HSC infusion. In some cases, a second dose of engineered hematopoietic stem cells may be administered to the recipient. In some cases, more than two doses of engineered hematopoietic stem cells are administered to the solid organ transplant recipient. Any of the above mentioned time frames may also pass between additional doses.

### Immunosuppression

Following the final dose of ATG administered to the recipient, prednisone can be administered. In some cases, a single dose of prednisone may be administered. In other cases, more than one dose of prednisone may be administered. For example, multiple doses of prednisone may be administered according to a tapering course or a constant course.

In some cases, for a tapering course, the first dose of prednisone may start at 100 mg/d and then the dose reduced by 5 mg/d until constant at 5 mg/d for at least 15 days, the first dose of prednisone may start at 90 mg/d and reduced by 5 mg/d until constant for at least 15 days, the first dose of prednisone may start at 80 mg/d and reduced by 5 mg/d until constant for at least 15 days, the first dose of prednisone may start at 70 mg/d and reduced by 5 mg/d until constant for at least 15 days, the first dose of prednisone may start at 60 mg/d and reduced by 5 mg/d until constant for at least 15 days, the first dose of prednisone may start at 50 mg/d and reduced by 5 mg/d until constant for at least 15 days, the first dose of prednisone may start at 40 mg/d and reduced by 5 mg/d until constant for at least 15 days, the first dose of prednisone may start at 30 mg/d and reduced by 5 mg/d until constant for at least 15 days, the first dose of prednisone may start at 20 mg/d and reduced by 5 mg/d until constant for at least 15 days or the first dose of prednisone may start at 10 mg/d and reduced by 5 mg/d until constant for at least 15 days. In some cases, for a constant course, the doses of prednisone may be 100 mg/d, 90 mg/d, 80 mg/d, 70 mg/d, 60 mg/d, 50 mg/d, 40 mg/d, 30 mg/d, 20 mg/d, 10 mg/d or 5 mg/d for at least 15 days.

In some cases, for a tapering course, the first dose of prednisone may start at 100 mg/d and reduced by 5 mg/d until constant for at least 30 days, the first dose of prednisone may start at 90 mg/d and reduced by 5 mg/d until constant for at least 30 days, the first dose of prednisone may start at 80 mg/d and reduced by 5 mg/d until constant for at least 30 days, the first dose of prednisone may start at 70 mg/d and reduced by 5 mg/d until constant for at least 30 days, the first dose of prednisone may start at 60 mg/d and reduced by 5 mg/d until constant for at least 30 days, the first dose of prednisone may start at 50 mg/d and reduced by 5 mg/d until constant for at least 30 days, the first dose of prednisone may start at 40 mg/d and reduced by 5 mg/d until constant for at least 30 days, the first dose of prednisone may start at 30 mg/d and reduced by 5 mg/d until constant for at least 30 days, the first dose of prednisone may start at 20 mg/d and reduced by 5 mg/d until constant for at least 30 days or the first dose of prednisone may start at 10 mg/d and reduced by 5 mg/d until constant for at least 30 days. In some cases, for a constant course, the doses of prednisone may be 100 mg/d, 90 mg/d, 80 mg/d, 70 mg/d, 60 mg/d, 50 mg/d, 40 mg/d, 30 mg/d, 20 mg/d, 10 mg/d or 5 mg/d for at least 30 days.

In some cases, for a tapering course, the first dose of prednisone may start at 100 mg/d and reduced by 5 mg/d until constant for at least 45 days, the first dose of prednisone may start at 90 mg/d and reduced by 5 mg/d until constant for at least 45 days, the first dose of prednisone may start at 80 mg/d and reduced by 5 mg/d until constant for at least 45 days, the first dose of prednisone may start at 70 mg/d and reduced by 5 mg/d until constant for at least 45 days, the first dose of prednisone may start at 60 mg/d and reduced by 5 mg/d until constant for at least 45 days, the first dose of prednisone may start at 50 mg/d and reduced by 5 mg/d until constant for at least 45 days, the first dose of prednisone may start at 40 mg/d and reduced by 5 mg/d until constant for at least 45 days, the first dose of prednisone may start at 30 mg/d and reduced by 5 mg/d until constant for at least 45 days, the first dose of prednisone may start at 20 mg/d and reduced by 5 mg/d until constant for at least 45 days or the first dose of prednisone may start at 10 mg/d and reduced by 5 mg/d until constant for at least 45 days. In some cases, for a constant course, the doses of prednisone may be 100 mg/d, 90 mg/d, 80 mg/d, 70 mg/d, 60 mg/d, 50 mg/d, 40 mg/d, 30 mg/d, 20 mg/d, 10 mg/d or 5 mg/d for at least 45 days.

In some cases, for a tapering course, the first dose of prednisone may start at 100 mg/d and reduced by 5 mg/d until constant for at least 60 days, the first dose of prednisone may start at 90 mg/d and reduced by 5 mg/d until constant for at least 60 days, the first dose of prednisone may start at 80 mg/d and reduced by 5 mg/d until constant for at least 60 days, the first dose of prednisone may start at 70 mg/d and reduced by 5 mg/d until constant for at least 60 days, the first dose of prednisone may start at 60 mg/d and reduced by 5 mg/d until constant for at least 60 days, the first dose of prednisone may start at 50 mg/d and reduced by 5 mg/d until constant for at least 60 days, the first dose of prednisone may start at 40 mg/d and reduced by 5 mg/d until constant for at least 60 days, the first dose of prednisone may start at 30 mg/d and reduced by 5 mg/d until constant for at least 60 days, the first dose of prednisone may start at 20 mg/d and reduced by 5 mg/d until constant for at least 60 days or the first dose of prednisone may start at 10 mg/d and reduced by 5 mg/d until constant for at least 60 days. In some cases, for a constant course, the doses of prednisone may be 100 mg/d, 90 mg/d, 80 mg/d, 70 mg/d, 60 mg/d, 50 mg/d, 40 mg/d, 30 mg/d, 20 mg/d, 10 mg/d or 5 mg/d for at least 60 days.

The corticosteroid and/or prednisone may be administered intravascularly, intravenously, intraarterially, intracranially, intraperitoneally, subcutaneously, intramuscularly, intraorbitally, orally, topically, or through any source which permits proper metabolism of the corticosteroid and/or prednisone by the recipient.

In some cases, recipients are treated with irradiation. The irradiation may be fractionated or unfractionated. In the case that a recipient is treated with more than one dose of irradiation, all doses may be fractionated. In another case that a recipient is treated with more than one dose of irradiation, all doses may be unfractionated. In another case that a recipient is treated with more than one dose of irradiation, the doses may be a mix of fractionated unfractionated.

In some cases, the irradiation is delivered intraoperatively. In some cases, the irradiation is delivered intravenously. In some cases, the irradiation is delivered intraarterially. In some cases, the irradiation is delivered subcutaneously. In some cases, the irradiation is delivered intraperitoneally.

In some cases, a single dose of irradiation may be delivered to the recipient. In other cases, the recipient may receive more than one dose of irradiation. For example, a recipient may receive at least one dose of irradiation, two doses of irradiation, three doses of irradiation, four doses of irradiation, five doses of irradiation, six doses of irradiation, seven doses of irradiation, eight doses of irradiation, nine doses of irradiation, 10 doses of irradiation, 11 doses of irradiation, 12 doses of irradiation, 13 doses of irradiation, 14 doses of irradiation, 15 doses of irradiation, 16 doses of irradiation, 17 doses of irradiation, 18 doses of irradiation, 19 doses of irradiation, or at least 20 doses of irradiation.

In some cases, each dose of irradiation may be at least 1 cGy, 2 cGy, 3 cGy, 4 cGy, 5 cGy, 6 cGy, 7 cGy, 8 cGy, 9 cGy, 10 cGy, 11 cGy, 12 cGy, 13 cGy, 14 cGy, 15 cGy, 16 cGy, 17 cGy, 18 cGy, 19 cGy, 20 cGy, 21 cGy, 22 cGy, 23 cGy, 24 cGy, 25 cGy, 26 cGy, 27 cGy, 28 cGy, 29 cGy, 30 cGy, 31 cGy, 32 cGy, 33 cGy, 34 cGy, 35 cGy, 36 cGy, 37 cGy, 38 cGy, 39 cGy, 40 cGy, 41 cGy, 42 cGy, 43 cGy, 44 cGy, 45 cGy, 46 cGy, 47 cGy, 48 cGy, 49 cGy, 50 cGy, 51 cGy, 52 cGy, 53 cGy, 54 cGy, 55 cGy, 56 cGy, 57 cGy, 58 cGy, 59 cGy, 60 cGy, 61 cGy, 62 cGy, 63 cGy, 64 cGy, 65 cGy, 66 cGy, 67 cGy, 68 cGy, 69 cGy, 70 cGy, 71 cGy, 72 cGy, 73 cGy, 74 cGy, 75 cGy, 76 cGy, 77 cGy, 78 cGy, 79 cGy, 80 cGy, 81 cGy, 82 cGy, 83 cGy, 84 cGy, 85 cGy, 86 cGy, 87 cGy, 88 cGy, 89 cGy, 90 cGy, 91 cGy, 92 cGy, 93 cGy, 94 cGy, 95 cGy, 96 cGy, 97 cGy, 98 cGy, 99 cGy, 100 cGy, 105 cGy, 110 cGy, 115 cGy, 120 cGy, 125 cGy, 130 cGy, 135 cGy, 140 cGy, 145 cGy, 150 cGy, 155 cGy, 160 cGy, 165 cGy, 170 cGy, 175 cGy, 180 cGy, 185 cGy, 190 cGy, 195 cGy, 200 cGy, 205 cGy, 210 cGy, 215 cGy, 220 cGy, 225 cGy, 230 cGy, 235 cGy, 240 cGy, 245 cGy, 250 cGy, 255 cGy, 260 cGy, 265 cGy, 270 cGy, 275 cGy, 280 cGy, 285 cGy, 290 cGy, 295 cGy, 300 cGy, 305 cGy, 310 cGy, 315 cGy, 320 cGy, 325 cGy, 330 cGy, 335 cGy, 340 cGy, 345 cGy, 350 cGy, 355 cGy, 360 cGy, 365 cGy, 370 cGy, 375 cGy, 380 cGy, 385 cGy, 390 cGy, 395 cGy, 400 cGy, 405 cGy, 410 cGy, 415 cGy, 420 cGy, 425 cGy, 430 cGy, 435 cGy, 440 cGy, 445 cGy, 450 cGy, 455 cGy, 460 cGy, 465 cGy, 470 cGy, 475 cGy, 480 cGy, 485 cGy, 490 cGy, 495 cGy or at least 500 cGy.

Irradiation may be administered on the same day of solid-organ transplantation. In some cases, the plurality of irradiation doses may be delivered over a period of time after organ transplantation. In some cases, the plurality of irradiation doses may be delivered over a period of at least 1 day, at least 2 days, at least 1 week, at least 2 week, 3 weeks, or more. In some cases, the doses of irradiation are delivered on a regular interval over the course of administration. In other cases, the doses of irradiation are not delivered on a regular interval over the course of administration. For example, irradiation may be delivered to the thymus gland on days 1 through 4, and days 7 through 11 after transplantation.

The irradiation may be targeted to a particular location of the recipient's body. In some cases, the irradiation may be targeted to a tissue, an organ, a region of the body or the whole body. In some cases, irradiation may be targeted to the lymph nodes, the spleen, or the thymus or any other area known to a person of skill in the art. In some cases, the irradiation may be targeted to the same location when at least more than one dose of irradiation is delivered to the patient. In other cases, the irradiation may be targeted a different location when at least more than one dose of irradiation is delivered to the patient.

During conditioning, recipients may be monitored for the development of conditions associated with non-myeloablative conditioning. Such diseases include neutropenia (e.g., granulocytes <2,000/mL), thrombocytopenia (e.g., platelets <60,000/mL) and secondary infections. In some cases, G-CSF (e.g., 10 μg/kg/day) may be administered for neutropenia. In some cases, any standard treatment known to one of skill in the art may be administered for thrombocytopenia or any secondary infections.

In some cases, conditioning may be temporarily stopped if a recipient develops neutropenia, thrombocytopenia or any secondary infections. Non-myeloablative conditioning may be continued once neutropenia, thrombocytopenia and or any secondary infections are resolved. In some cases, if the recipient has a white blood count below 1,000 cells/mm³, the recipient may be treated with G-CSF (e.g., 10 μg/kg/day) following non-myeloablative conditioning.

### Immunosuppression and Graft Management

Following either HLA-matched or HLA-mismatched solid organ transplantation and administration of the engineered HLA-matched or HLA-mismatched hematopoietic cells, the recipient may receive an immunosuppressive regimen. The immunosuppressive regimen may have two phases, an induction phase and a maintenance phase. Induction and maintenance phase strategies may use different medicines at doses adjusted to achieve target therapeutic levels to enhance long term transplant persistence in the recipient. In some cases, the induction phase may begin perioperatively. In some cases, the induction phase may begin immediately after transplantation. In some cases, the induction phase may be both perioperative and immediately after transplantation. In some cases, the immunosuppressive regimen may continue as a maintenance therapy until the recipient achieves chimerism. For example, chimerism may be stable mixed chimerism as described herein.

In some cases, the immunosuppressive regimen may include one agent. In other cases, the immunosuppressive regimen may include more than one agent. For example, suitable agents for the immunosuppressive regimen may include a calcineurin inhibitor and/or an adjuvant. In some cases, the primary immunosuppressive agents include calcineurin inhibitors, which combine with binding proteins to inhibit calcineurin activity. In some cases, the calcineurin inhibitor may be tacrolimus, cyclosporine A, or any calcineurin inhibitor known to one of skill in the art and may be administered to the recipient at a dose effective to provide targeted immunosuppression as a calcineurin inhibitor.

In some cases, cyclosporine A may be withdrawn from the recipient after a duration of less than one month, two months, three months, four months, five months, six months, seven months, eight months, nine months, ten months, 11 months, 12 months, 13 months, 14 months, 15 months, 16 months, 17 months, 18 months, 19 months, 20 months, 21 months, 22 months, 23 months or less than 24 months.

In some cases, cyclosporine A may be withdrawn from the recipient after a duration of more than one month, two months, three months, four months, five months, six months, seven months, eight months, nine months, ten months, 11 months, 12 months, 13 months, 14 months, 15 months, 16 months, 17 months, 18 months, 19 months, 20 months, 21 months, 22 months, 23 months or more than 24 months.

In some cases, the dose of cyclosporine A may slowly be tapered if the recipient meets clinical criteria for lack of rejection and GVHD. For example, the total amount of the cyclosporine A administered may be reduced over time. In some cases, tapering of the cyclosporine A may occur for a duration of less than one month, two months, three months, four months, five months, six months, seven months, eight months, nine months, ten months, 11 months, 12 months, 13 months, 14 months, 15 months, 16 months, 17 months, 18 months, 19 months, 20 months, 21 months, 22 months, 23 months or less than 24 months such that at the end of the tapering regime, the dose of the cyclosporine A is tapered to zero. In some cases, tapering of the cyclosporine A may occur for a duration of more than one month, two months, three months, four months, five months, six months, seven months, eight months, nine months, ten months, 11 months, 12 months, 13 months, 14 months, 15 months, 16 months, 17 months, 18 months, 19 months, 20 months, 21 months, 22 months, 23 months or more than 24 months such that at the end of the tapering regime, the dose of the cyclosporine A is tapered to zero.

In some cases, the cyclosporine A may be delivered by a single dose to the recipient. In other cases, the recipient may receive more than one dose of cyclosporine A. For example, a recipient may receive at least one dose of cyclosporine A, two doses of cyclosporine A, three doses of cyclosporine A, four doses of cyclosporine A, five doses of cyclosporine A, six doses of cyclosporine A, seven doses of cyclosporine A, eight doses of cyclosporine A, nine doses of cyclosporine A, 10 doses of cyclosporine A, 11 doses of cyclosporine A, 12 doses of cyclosporine A, 13 doses of cyclosporine A, 14 doses of cyclosporine A, 15 doses of cyclosporine A, 16 doses of cyclosporine A, 17 doses of cyclosporine A, 18 doses of cyclosporine A, 19 doses of cyclosporine A, or 20 doses of cyclosporine A.

In some cases, a plurality of cyclosporine A doses may be delivered over a period of time after organ transplantation. In some cases, the plurality of cyclosporine A doses may be delivered over a period of at least 0.1 days, 0.2 days, 0.3 days, 0.4 days, 0.5 days, 0.6 days, 0.7 days, 0.8 days, 0.9 days, 1.0 days, 1.1 days, 1.2 days, 1.3 days, 1.4 days, 1.5 days, 1.6 days, 1.7 days, 1.8 days, 1.9 days, 2.0 days, 2.1 days, 2.2 days, 2.3 days, 2.4 days, 2.5 days, 2.6 days, 2.7 days, 2.8 days, 2.9 days, 3.0 days, 3.1 days, 3.2 days, 3.3 days, 3.4 days, 3.5 days, 3.6 days, 3.7 days, 3.8 days, 3.9 days, 4.0 days, 4.1 days, 4.2 days, 4.3 days, 4.4 days, 4.5 days, 4.6 days, 4.7 days, 4.8 days, 4.9 days, 5.0 days, 5.1 days, 5.2 days, 5.3 days, 5.4 days, 5.5 days, 5.6 days, 5.7 days, 5.8 days, 5.9 days, 6.0 days, 6.1 days, 6.2 days, 6.3 days, 6.4 days, 6.5 days, 6.6 days, 6.7 days, 6.8 days, 6.9 days, 7.0 days, 7.1 days, 7.2 days, 7.3 days, 7.4 days, 7.5 days, 7.6 days, 7.7 days, 7.8 days, 7.9 days, 8.0 days, 8.1 days, 8.2 days, 8.3 days, 8.4 days, 8.5 days, 8.6 days, 8.7 days, 8.8 days, 8.9 days, 9.0 days, 9.1 days, 9.2 days, 9.3 days, 9.4 days, 9.5 days, 9.6 days, 9.7 days, 9.8 days, 9.9 days, 10 days, 10.5 days, 11 days, 11.5 days, 12 days, 12.5 days, 13 days, 13.5 days, 14 days, 14.5 days, 15 days, 15.5 days, 16 days, 16.5 days, 17 days, 17.5 days, 18 days, 18.5 days, 19 days or at least 20 days.

In some cases, each dose of cyclosporine A may be at least 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6.0 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 7.0 mg/kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8.0 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 9.0 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, or at least 10 mg/kg.

In some cases, the amount of cyclosporine A administered to the patient may be determined by the amount of the cyclosporine A in the bloodstream. For example, the cyclosporine A may be administered at a dose to achieve a range of 0-40 mg, 5-50 mg, 10-60 mg, 15-65 mg, 20-70 mg, 25-75 mg, 30-80 mg, 35-85 mg, 40-90 mg, 45-95 mg, 50-100 mg, 55-105 mg, 60-110 mg, 65-115 mg, 70-120 mg, 75-125 mg, 80-130 mg, 85-135 mg, 90-140 mg, 95-145 mg, 100-150 mg, 105-155 mg, 110-160 mg, 115-165 mg, 120-170 mg, 125-175 mg, 130-180 mg, 135-185 mg, 140-190 mg, 145-195 mg, 150-200 mg, 160-210 mg, 170-220 mg, 180-230 mg, 190-240 mg, 200-250 mg, 210-260 mg, 220-270 mg, 230-280 mg, 240-290 mg, 250-300 mg, 260-310 mg, 270-320 mg, 280-330 mg, 290-340 mg, 300-350 mg, 310-360 mg, 320-370 mg, 330-380 mg, 340-390 mg, 350-400 mg, 360-410 mg, 370-420 mg, 380-430 mg, 390-440 mg, 400-450 mg, 410-460 mg, 420-470 mg, 430-480 mg, 440-490 mg, 450-500 mg, 46-510 mg, 470-520 mg, 480-530 mg, 490-540 mg, 500-550 mg, 510-560 mg, 520-570 mg, 530-580 mg, 540-590 mg, 550-600 mg, 560-610 mg, 570-620 mg, 580-630 mg, 590-640 mg, 600-650 mg, 610-660 mg, 620-670 mg, 630-680 mg, 640-690 mg, 650-700 mg or more than 700 mg.

In some cases, tacrolimus may be withdrawn from the recipient after a duration of more than one month, two months, three months, four months, five months, six months, seven months, eight months, nine months, ten months, 11 months, 12 months, 13 months, 14 months, 15 months, 16 months, 17 months, 18 months, 19 months, 20 months, 21 months, 22 months, 23 months or more than 24 months. In some cases, the dose of tacrolimus may slowly be tapered providing the recipient meets clinical criteria for lack of rejection and GVHD. For example, the total amount of tacrolimus administered may be reduced over time. In some cases, tapering of tacrolimus may occur for a duration of less than one month, two months, three months, four months, five months, six months, seven months, eight months, nine months, ten months, 11 months, 12 months, 13 months, 14 months, 15 months, 16 months, 17 months, 18 months, 19 months, 20 months, 21 months, 22 months, 23 months or less than 24 months such that at the end of the tapering regime, the dose of tacrolimus is tapered to zero.

In some cases, tacrolimus may be withdrawn from the recipient after a duration of less than one month, two months, three months, four months, five months, six months, seven months, eight months, nine months, ten months, 11 months, 12 months, 13 months, 14 months, 15 months, 16 months, 17 months, 18 months, 19 months, 20 months, 21 months, 22 months, 23 months or less than 24 months. In some cases, the dose of tacrolimus may slowly be tapered providing the recipient meets clinical criteria for lack of rejection and GVHD. For example, the total amount of tacrolimus administered may be reduced over time. In some cases, tapering of tacrolimus may occur for a duration of more than one month, two months, three months, four months, five months, six months, seven months, eight months, nine months, ten months, 11 months, 12 months, 13 months, 14 months, 15 months, 16 months, 17 months, 18 months, 19 months, 20 months, 21 months, 22 months, 23 months or more than 24 months such that at the end of the tapering regime, the dose of tacrolimus is tapered to zero.

In some cases, tacrolimus may be delivered by a single to the recipient. In other cases, the recipient may receive more than one dose of Tacrolimus. For example, a recipient may receive at least one dose of Tacrolimus, two doses of Tacrolimus, three doses of Tacrolimus, four doses of Tacrolimus, five doses of Tacrolimus, six doses of Tacrolimus, seven doses of Tacrolimus, eight doses of Tacrolimus, nine doses of Tacrolimus, 10 doses of Tacrolimus, 11 doses of Tacrolimus, 12 doses of Tacrolimus, 13 doses of Tacrolimus, 14 doses of Tacrolimus, 15 doses of Tacrolimus, 16 doses of Tacrolimus, 17 doses of Tacrolimus, 18 doses of Tacrolimus, 19 doses of Tacrolimus, or at least 20 doses of Tacrolimus.

In some cases, a plurality of tacrolimus doses may be delivered over a period of time after organ transplantation. In some cases, the plurality of tacrolimus doses may be delivered over a period of at least 0.1 days, 0.2 days, 0.3 days, 0.4 days, 0.5 days, 0.6 days, 0.7 days, 0.8 days, 0.9 days, 1.0 days, 1.1 days, 1.2 days, 1.3 days, 1.4 days, 1.5 days, 1.6 days, 1.7 days, 1.8 days, 1.9 days, 2.0 days, 2.1 days, 2.2d days, 2.3 days, 2.4 days, 2.5 days, 2.6 days, 2.7 days, 2.8 days, 2.9 days, 3.0 days, 3.1 days, 3.2 days, 3.3 days, 3.4 days, 3.5 days, 3.6 days, 3.7 days, 3.8 days, 3.9 days, 4.0 days, 4.1 days, 4.2 days, 4.3 days, 4.4 days, 4.5 days, 4.6 days, 4.7 days, 4.8 days, 4.9 days, 5.0 days, 5.1 days, 5.2 days, 5.3 days, 5.4 days, 5.5 days, 5.6 days, 5.7 days, 5.8 days, 5.9 days, 6.0 days, 6.1 days, 6.2 days, 6.3 days, 6.4 days, 6.5 days, 6.6 days, 6.7 days, 6.8 days, 6.9 days, 7.0 days, 7.1 days, 7.2 days, 7.3 days, 7.4 days, 7.5 days, 7.6 days, 7.7 days, 7.8 days, 7.9 days, 8.0 days, 8.1 days, 8.2 days, 8.3 days, 8.4 days, 8.5 days, 8.6 days, 8.7 days, 8.8 days, 8.9 days, 9.0 days, 9.1 days, 9.2 days, 9.3 days, 9.4 days, 9.5 days, 9.6 days, 9.7 days, 9.8 days, 9.9 days, 10 days, 10.5 days, 11 days, 11.5 days, 12 days, 12.5 days, 13 days, 13.5 days, 14 days, 14.5 days, 15 days, 15.5 days, 16 days, 16.5 days, 17 days, 17.5 days, 18 days, 18.5 days, 19 days or at least 20 days.

In some cases, each dose of tacrolimus may be at least 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6.0 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 7.0 mg/kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8.0 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 9.0 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, or at least 10 mg/kg.

In some cases, the amount of tacrolimus administered to the patient is determined by the amount of tacrolimus in the bloodstream. For example, tacrolimus may be administered at a dose to achieve a range of 0-40 mg, 5-50 mg, 10-60 mg, 15-65 mg, 20-70 mg, 25-75 mg, 30-80 mg, 35-85 mg, 40-90 mg, 45-95 mg, 50-100 mg, 55-105 mg, 60-110 mg, 65-115 mg, 70-120 mg, 75-125 mg, 80-130 mg, 85-135 mg, 90-140 mg, 95-145 mg, 100-150 mg, 105-155 mg, 110-160 mg, 115-165 mg, 120-170 mg, 125-175 mg, 130-180 mg, 135-185 mg, 140-190 mg, 145-195 mg, 150-200 mg, 160-210 mg, 170-220 mg, 180-230 mg, 190-240 mg, 200-250 mg, 210-260 mg, 220-270 mg, 230-280 mg, 240-290 mg, 250-300 mg, 260-310 mg, 270-320 mg, 280-330 mg, 290-340 mg, 300-350 mg, 310-360 mg, 320-370 mg, 330-380 mg, 340-390 mg, 350-400 mg, 360-410 mg, 370-420 mg, 380-430 mg, 390-440 mg, 400-450 mg, 410-460 mg, 420-470 mg, 430-480 mg, 440-490 mg, 450-500 mg, 46-510 mg, 470-520 mg, 480-530 mg, 490-540 mg, 500-550 mg, 510-560 mg, 520-570 mg, 530-580 mg, 540-590 mg, 550-600 mg, 560-610 mg, 570-620 mg, 580-630 mg, 590-640 mg, 600-650 mg, 610-660 mg, 620-670 mg, 630-680 mg, 640-690 mg, 650-700 mg or more than 700 mg.

The levels of either cyclosporine or tacrolimus in the recipient may be monitored. At the onset of immunosuppression, the levels of either cyclosporine or tacrolimus may be, for example, in the range of 0-15 ng/mL, 5-15 ng/mL, 10-20 ng/mL, 15-25 ng/mL, 20-30 ng/mL, 25-35 ng/mL, 30-40 ng/mL, 35-45 ng/mL or 40-50 ng/mL in the recipient. In some cases, the levels of either cyclosporine or tacrolimus may be reduced after a period of time in the recipient. For example, the period of time may be less than one week, two weeks, three weeks, four weeks, five weeks, six weeks, seven weeks, eight weeks, nine weeks, ten weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks or less than 33 weeks. In some cases, the levels of either cyclosporine or tacrolimus may be reduced to within the range of 0-1 ng/mL, 0.5-1.5 ng/mL, 1.0-2.0 ng/mL, 1.5-2.5 ng/mL, 2.0-3.0 ng/mL, 2.5-3.5 ng/mL, 3.0-4.0 ng/mL, 3.5-4.5 ng/mL, 4.0-5.0 ng/mL, 5.5-6.5 ng/mL, 6.0-7.0 ng/mL, 6.5-7.5 ng/mL, 7.0-8.0 ng/mL, 8.5-9.5 ng/mL or 9.0-10.0 ng/mL in the recipient.

In some cases, a calcineurin inhibitor may be administered to the recipient in combination with an inhibitor of purine metabolism (e.g., mycophenolate mofetil). For example, cyclosporine A and mycophenolate mofetil may be used in the case of kidney transplantation.

In some cases, the adjuvant may be withdrawn from the recipient after a duration of more than one month, two months, three months, four months, five months, six months, seven months, eight months, nine months, ten months, 11 months, 12 months, 13 months, 14 months, 15 months, 16 months, 17 months, 18 months, 19 months, 20 months, 21 months, 22 months, 23 months or more than 24 months. In some cases, the dose of the adjuvant may slowly be tapered providing the recipient meets clinical criteria for lack of rejection and GVHD. For example, the total amount of the adjuvant administered may be reduced over time. In some cases, tapering of the adjuvant may occur for a duration of more than one month, two months, three months, four months, five months, six months, seven months, eight months, nine months, ten months, 11 months, 12 months, 13 months, 14 months, 15 months, 16 months, 17 months, 18 months, 19 months, 20 months, 21 months, 22 months, 23 months or more than 24 months such that at the end of the tapering regime, the dose of the purine metabolism inhibitor is tapered to zero.

In some cases, the adjuvant may be withdrawn from the recipient after a duration of less than one month, two months, three months, four months, five months, six months, seven months, eight months, nine months, ten months, 11 months, 12 months, 13 months, 14 months, 15 months, 16 months, 17 months, 18 months, 19 months, 20 months, 21 months, 22 months, 23 months or less than 24 months. In some cases, the dose of the adjuvant may slowly be tapered providing the recipient meets clinical criteria for lack of rejection and GVHD. For example, the total amount of the adjuvant administered may be reduced over time. In some cases, tapering of the adjuvant may occur for a duration of less than one month, two months, three months, four months, five months, six months, seven months, eight months, nine months, ten months, 11 months, 12 months, 13 months, 14 months, 15 months, 16 months, 17 months, 18 months, 19 months, 20 months, 21 months, 22 months, 23 months or less than 24 months such that at the end of the tapering regime, the dose of the purine metabolism inhibitor is tapered to zero.

Adjuvant agents may be used to enhance immunosuppression while decreasing the dose and toxicity of other individual agents that are part of the immunosuppressive regimen. In some cases, adjuvant agents may be combined with a calcineurin inhibitor. For example, adjuvant agents may include steroids, azathioprine, mycophenolate mofetil, sirolimus, an antibody or any adjuvant agent known to one of skill in the art and may be administered to the recipient at a dose effective to enhance immunosuppression.

In some cases, antibody-based therapy may be used to avoid or reduce the dose of calcineurin inhibitors in the immunosuppressive regimen. For example, antibody-based therapy may include monoclonal (e.g., muromonab-CD3) antibodies, polyclonal antibodies and/or anti-CD25 antibodies (eg, basiliximab, daclizumab). In some cases, antibody-based therapy may be administered during the early post-transplant period. For example, early post-transplant may be up to 8 weeks following the transplant.

Graft management may include preventing, inhibiting or suppressing acute rejection with immunosuppressive drugs. In some cases, multiple agents may be used to prevent, inhibit or suppress episodes of acute rejection. For example, an agent may be a steroid. In some cases, one or more than one steroid may be used to prevent, inhibit or suppress episodes of acute rejection. Any steroid known to one of skill in the art suitable for preventing, inhibiting or suppressing acute rejection may be used. For example, any dose, mode of administration and duration of administration for any steroid known to one of skill in the art suitable for preventing, inhibiting or suppressing acute rejection may be used. In some cases, administration of the steroid may be tapered to a maintenance dose.

For example, an agent may be antithymocyte globulin. In some cases, antithymocyte globulin may be used to prevent, inhibit or suppress episodes of acute rejection. Any dose, mode of administration and duration of administration for antithymocyte globulin suitable for preventing, inhibiting or suppressing acute rejection may be used. In some cases, administration of antithymocyte globulin may be tapered to a maintenance dose.

For example, an agent may be muromonab-CD3. In some cases, muromonab-CD3 may be used to prevent, inhibit or suppress episodes of acute rejection. Any dose, mode of administration and duration of administration for muromonab-CD3 suitable for preventing, inhibiting or suppressing acute rejection may be used. In some cases, administration of muromonab-CD3 may be tapered to a maintenance dose.

### Chimerism

Following either HLA-matched or HLA-mismatched solid organ transplantation and administration of the engineered HLA-matched or HLA-mismatched hematopoietic cells, the recipient may be monitored for chimerism. Recipients who exhibit greater than 95% donor cells in a given blood cell lineage by any analysis to determine chimerism at any time post-transplantation may be classified as having full donor chimerism. In some cases, mixed chimerism may be greater than 1% donor-derived cells of a given lineage but less than 95% donor-derived DNA.

Individuals who exhibit mixed chimerism may be further classified according to the evolution of chimerism, where improving mixed chimerism may be a continuous increase in the proportion of donor cells over a period of time (e.g., at least a 6-months). In some cases, stable mixed chimerism may include fluctuations in the percentage of recipient cells over time, without complete loss of donor cells.

Mixed chimerism may be determined by measuring the percentage of donor cells for a single cell type within the recipient. For example, mixed chimerism may be determined by the percentage of donor-derived granulocytes in the recipient. In some cases, mixed chimerism may be determined by measuring the percentage of donor cells for a plurality of cell types within the recipient. For example, mixed chimerism may be determined by the percentage of donor-derived granulocytes, natural killer cells, B cells and T cells in the recipient.

There are a plurality of methods of testing for chimerism that are readily available and known to those of skill in the art. Any method of testing for chimerism that distinguishes donor or recipient origin of a cell is suitable for use in the methods described herein.

In some cases, the methods of testing for chimerism may include genetic based methods. For example, polymerase chain reaction (PCR) based methods which utilize probes may be used. In some cases, probes for PCR based methods may be probes for microsatellite analysis. For another example, commercial kits that distinguish polymorphisms in short terminal repeat lengths of donor and host origin are readily available and known to those of skill in the art.

In some cases, major histocompatibility complex (MHC) typing may be used for testing chimerism. For example, MHC typing may be used to test the type of cells in the blood. In some cases, MHC typing may be used in combination with flow cytometry. In some case, an analysis of HLA-stained cells by flow cytometry may be performed.

The methods described herein are provided such that a recipient may achieve stable mixed chimerism sufficient to allow withdrawal of immunosuppressive drugs. For example, withdrawal of immunosuppressive drugs may include tapering immunosuppressive drugs. In other cases, withdrawal of immunosuppressive drugs may include immediate withdrawal of immunosuppressive drugs. In some cases, mixed chimerism persists for at least six months prior to withdrawal of immunosuppressive drugs. In other cases, mixed chimerism persists for at least one month, two months, three months, four months, five months, six months, seven months, eight months, nine months, ten months, 11 months, 12 months, 13 months, 14 months, 15 months, 16 months, 17 months, 18 months, 19 months, 20 months, 21 months, 22 months, 23 months or at least 24 months. In some cases, the dose of the adjuvant may slowly be tapered providing the recipient meets clinical criteria for lack of rejection and GVHD. For example, the total amount of the adjuvant administered may be reduced over time. In some cases, tapering of the adjuvant may occur for a duration of at least one month, two months, three months, four months, five months, six months, seven months, eight months, nine months, ten months, 11 months, 12 months, 13 months, 14 months, 15 months, 16 months, 17 months, 18 months, 19 months, 20 months, 21 months, 22 months, 23 months or at least 24 months.

In some cases, a lack of rejection episodes may coincide with mixed chimerism prior to withdrawal of immunosuppressive drugs. In some cases, a lack of rejection episodes may be consistent for at least six months prior to withdrawal of immunosuppressive drugs. In other cases, a lack of rejection episodes may be consistent for at least one month, two months, three months, four months, five months, six months, seven months, eight months, nine months, ten months, 11 months, 12 months, 13 months, 14 months, 15 months, 16 months, 17 months, 18 months, 19 months, 20 months, 21 months, 22 months, 23 months or at least 24 months. In some cases, the dose of the adjuvant may slowly be tapered providing the recipient meets clinical criteria for lack of rejection and GVHD. For example, the total amount of the adjuvant administered may be reduced over time. In some cases, tapering of the adjuvant may occur for a duration of at least one month, two months, three months, four months, five months, six months, seven months, eight months, nine months, ten months, 11 months, 12 months, 13 months, 14 months, 15 months, 16 months, 17 months, 18 months, 19 months, 20 months, 21 months, 22 months, 23 months or at least 24 months.

In some cases, a lack of GVHD and lack of rejection episodes coincides with mixed chimerism prior to withdrawal of immunosuppressive drugs. In some cases, a lack of GVHD and lack of rejection episodes may be consistent for at least six months prior to withdrawal of immunosuppressive drugs. In other cases, a lack of GVHD and lack of rejection episodes may be consistent for at least one month, two months, three months, four months, five months, six months, seven months, eight months, nine months, ten months, 11 months, 12 months, 13 months, 14 months, 15 months, 16 months, 17 months, 18 months, 19 months, 20 months, 21 months, 22 months, 23 months or at least 24 months. In some cases, the dose of the adjuvant may slowly be tapered providing the recipient meets clinical criteria for lack of rejection and GVHD. For example, the total amount of the adjuvant administered may be reduced over time. In some cases, tapering of the adjuvant may occur for a duration of at least one month, two months, three months, four months, five months, six months, seven months, eight months, nine months, ten months, 11 months, 12 months, 13 months, 14 months, 15 months, 16 months, 17 months, 18 months, 19 months, 20 months, 21 months, 22 months, 23 months or at least 24 months.

In order to determine if tapering of the immunosuppressive regimen is appropriate for the recipient, the recipient may be tested for mixed chimerism, usually at regular intervals. For example, regular intervals may be monthly, semi-monthly, weekly, bi-monthly, annually, bi-annually or the like.

The invention now being fully described, it is apparent to one of ordinary skill in the art that various changes and modifications can be made without departing from the spirit or scope of the invention.

### EXAMPLES

The present disclosure has been described in terms of particular cases found or proposed to comprise preferred modes for the practice of the disclosure. It is appreciated by those of skill in the art that, in light of the present disclosure, numerous modifications and changes can be made in the particular embodiments exemplified without departing from the intended scope of the disclosure. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

For further elaboration of general techniques useful in the practice of this disclosure, the practitioner can refer to standard textbooks and reviews in cell biology, tissue culture, and embryology. With respect to tissue culture and embryonic stem cells, the reader may wish to refer to Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in Vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Fertil. Dev. 10:31, 1998).

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The following examples are put forth so as to provide those of ordinary skill in the art with a complete disclosure and description of how to make and use the present disclosure and are not intended to limit the scope of what is regarded as the disclosure nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Persistent donor hematopoietic cell chimerism using host conditioning with total lymphoid irradiation (TLI) combined with a single, very low dose of total body irradiation (svldTBI) and the infusion of donor hematopoietic cell subsets for organ and tissue transplantation and autoimmune tolerance.

### Regimen

We describe a significant and novel non-intuitive improvement to the current recipient conditioning regimen that is used induce persistent mixed chimerism in HLA matched living related donor kidney transplants.

Host conditioning with a single fraction (one dose) of TBI is commonly used in combination with fludarabine and/or alkylating chemotherapy agents as has been described in cancer patients undergoing BMT for decades. In these published studies TBI was dosed between 200-400 cGy and this created marrow space and induced significant marrow hypoplasia and cytopenias such that virtually all patients developed profound neutropenia, thrombocytopenia, and anemia with a requirement for blood and platelet transfusion support for more than 2 weeks. As a result of the profound marrow hypoplasia from the TBI-based recipient conditioning complete donor cell chimerism occurred, and the donor cell graft in these studies functioned as a replacement marrow following TBI-based conditioning. It is well established that 200-400 cGy TBI-based recipient conditioning is associated with advanced acute GVHD in about 20-40% of recipients, and chronic GVHD in about 30% recipients. In the public domain there is a recipient conditioning using TBI 200 cGy combined with fludarabine and cyclophosphamide for kidney transplant organ tolerance induction involving living donors only yet as expected this regimen results in complete donor cell chimerism (not mixed chimerism) and is associated with acute and chronic GVHD.

Here, we describe a non-obvious, new recipient conditioning: TLI-ATG will be administered in the regular manner yet one dose of TLI will be omitted, and instead a single, very low dose of TBI (svldTBI, 40-140 cGy, much lower than ever considered helpful or useful) is administered. Currently and despite decades of using TBI for recipient BMT conditioning, a single TBI dose of less than 200 cGy has not been administered to humans, in part, because a single dose less than 200 cGy is not expected to induce enough marrow hypoplasia to facilitate donor cell engraftment and chimerism. In the current application, the svldTBI (40-140 cGy) is also not expected to induce marrow hypoplasia, rather the svldTBI is expected to provide enhanced host lympho-depletion and without increasing recipient organ toxicity owning to the single very low dose. Unlike TLI, TBI does not shield the gut, liver and lungs, and consequently the large immune cell reservoirs residing within these organs will be partially depleted following the single, very low dose of irradiation. The enhanced non-lymphoid immune cell depletion is expected to remove resistance to donor cell engraftment, and allow persistent mixed chimerism following infusions of hematopoietic cells from living related and unrelated donors with all degrees of HLA mismatch, and from deceased donors. The svldTBI is not expected to induce significant marrow hypoplasia, cytopenia, or GI toxicity. The TLI-svldTBI-ATG regimen is expected to protect against GVHD as mixed chimerism is protective. We performed TLI-svldTBI-ATG host conditioning in cancer and renal tolerance patients and as predicted have not observed cytopenias, or incurred GI toxicity (unpublished observations from August-December 2019).

### Composition

The current hematopoietic cell composition is insufficient to achieve persistent mixed donor cell chimerism in organ transplant recipients from living related and unrelated donors of all degrees of HLA mismatch, and from deceased donors. We herein describe a novel composition of matter of a donor cell product that facilitates persistent mixed chimerism and allow IS drug minimization and/or complete drug cessation following combined organ (kidney, heart, liver, lungs, and bowel), tissue and composite tissue and hematopoietic cell transplants from living related and unrelated donors of all degrees of HLA mismatch, and from deceased donors. The donor cell inoculum described herein when combined with the unique TLI-svldTBI-ATG recipient conditioning is expected to result in persistent mixed chimerism that is a requirement for transplantation tolerance and a requirement for GVHD protection.

In the case of living HLA mismatched related and unrelated donors: donor hematopoietic cells will be mobilized using granulocyte colony stimulating factor (G-CSF)+/−mozobil, and the donor will undergo 1 or 2 consecutive days of high volume (>12 liters) blood apheresis to obtain blood mononuclear cells in the usual manner as per standard of care for BMT donors in cancer patients. The apheresis collection(s) will be processed for CD34+ cell enrichment using either fluorescence activated cell sorting (FACS) or magnetic activated cell sorting (MACS) as per manufacturer's guidelines. The CD34+ enriched product will be cryopreserved in the standard manner. The pre-freeze CD34+ cell purity must be ≥70%. The CD34+ cell dose will be a pre-freeze value of 8-20 million CD34+ cells/kg recipient weight.

In some cases, the non-CD34+ cell fraction following the MACS or FACS CD34+ enrichment step will be used to obtain a defined dose of CD3+ T cells (a pre-freeze dose of 25-100 million CD3+/kg recipient weight), and will be cryopreserved in the usual manner.

In some cases, enriched populations of donor derived CD8+ memory T cells (defined as CD3+/CD8+/CD45RA−/CD45RO+ and enrichment methods described in U.S. Pat. No. 9,833,477 B2, issued Dec. 5, 2017 which is for use in cancer patients) at a dose of 1-12 million/kg may be infused 0-3 days after the CD34+ enriched cell product and in place of donor CD3+ T cells.

In some cases, donor derived Treg cells (CD4⁺CD25⁺FoxP3⁺) enriched by FACS or MACS methods will be infused 0-4 days after the infusion of donor CD34+ enriched cells at a dose of 1-10 million/kg.

In some cases, the donor Treg cells will be combined with donor CD3+ T cells in a non-intuitive and non-physiologic ratio of Treg:CD3+ T cells ranging from 1:50 to 3:1 and infused 0-4 days after the infusion of donor CD34+ enriched cells.

All of the above described donor cell inoculums represent adjustments of naturally occurring physiologic cell ratios and represent the intellectual property of the composition of matter in donor graft manipulation. The CD34+ enriched cells when combined with CD3+ T cell and/or CD8+ memory T cells and/or Treg cells will be expected to result in persistent mixed donor cell chimerism following the unique recipient conditioning of TLI-svldTBI-ATG.

In the cases of deceased donor organs, we will obtain deceased donor hematopoietic and immune cells from vertebral bodies (VBs), pelvic bones, and spleen and cryopreserve the cells in the usual manner. The deceased donor hematopoietic and immune cells will be thawed and infused into the recipient following host TLI-svldTBI-ATG conditioning. This will be the first in-human application of a host conditioning regimen combined with a uniquely defined hematopoietic and immune cell product to establish persistent mixed donor cell chimerism using cells obtained from deceased donors. Persistent mixed chimerism will lead to organ transplantation tolerance, and IS drug minimization and/or withdrawal.

To obtain deceased donor bone marrow cells from the VBs we transect the VB at the vertebral arch and in a unique procedural step apply a razor thin high-pressure saline jet stream to “power-wash” away connective tissue and necrotic surgical/bacterial/cellular debris from the VB. After the VB is power-washed, a rotary saw slices open the VBs and pea sized chunks are subsequently made. Taken together these methods maximize the VB bone marrow surface area that allows maximum cell extraction and yield. The cell product is passed through a multi-sieve elution and purification step. These novel methods significantly improve VB cell yields and purity compared to previously published procedures and methods.

Using VB bone marrow cells the CD34+ cell dose range will be 2-20 million/kg recipient weight and the CD3+ T cell dose range will be 10-100 million/kg.

In some instances we will obtain splenocytes to supplement the VB bone marrow cell inoculum. We previously determined (unpublished data on file) that several (typically 2-8) 2 inch-sized splenic cubes removed from the donor spleen will be needed as a supplemental immune cell source to support persistent mixed donor cell chimerism. The splenic cubes will be harvested during the time of organ procurement and transported in standard transport media along with the donor VBs. A single cell suspension consisting of live mononuclear splenocytes will be obtained by dissociating the cells from the splenic tissue using a specialized dissociation media and techniques to prevent i) over-digestion by chemical and proteolytic enzymes, and ii) excessive tissue disaggregation from environmental stress by excessive mechanical forces, vortexing, homogenization, abnormal osmolality stresses or combinations thereof. The single cell suspension will be passed through a multi-sieve elution tower with a final 80-120 micron strainer. The cell pellet will be prepared for cryopreservation with or without MACS/FACS separation of the live cells for aliquots of CD3+ cells, and Treg cells, mesenchymal stem cells (MSCs), B cells, invariant natural killer (iNK) cells and hematopoietic cell precursors. These cell types can be used in cell expansion protocols which may allow for the treatment of one or more recipients.

### Use of Splenic Cells:

In some cases the splenic CD3+ T cells will be added to the infused VB bone marrow cells to augment the donor CD3 T cell dose if it is low (for example if less than 50 million CD3+ T cells are obtained from the VB bone marrow cells).

In some cases, the splenic T cells will be added to enable CD3+ T cells doses that may be as high as 200 million CD3+ T cells/kg.

In some cases the splenocytes will be used to exclusively obtain Treg cells to be used in doses of 1-10 million/kg recipient weight.

In some cases the splenic Treg cells may be engineered with a predetermined antigen-specificity via transfection of viral vectors encoding specific T cell receptors (TCRs) or chimeric antigen receptors (CARs). The engineered Treg cells may express tissue specific antigens that promote Treg cells trafficking, migrating and residing in selected recipient tissues (bone marrow, lymph nodes, neuronal, heart, lung, kidney, liver, bowel, and pancreas) to promote local immune suppressive reactions that enhance persistent mixed chimerism and/or tissue-specific tolerance. Treg may be used as primary cells or in culture expansion and potentially in multiple recipients.

In some cases, a “left over” fraction of the VB bone marrow and/or splenocytes may be cryopreserved and stored for months to years, and can be given as a later donor cell boost if chimerism and/or tolerance is waning over time.

In some cases, use of deceased donor bone marrow and spleen cell subsets as outlined above will be infused into recipients with relapsing and refractory autoimmune disorders to establish immune regulation and tolerance and provide durable autoimmune disease control.

Taken together we herein describe a new recipient transplant tolerance conditioning regimen that involves 9 doses of TLI and one, non-obvious, very low dose of TBI combined with ATG. The TBI dose in the new regimen is far lower than any previously published single dose TBI. The single, and very low dose of TBI is not expected to induce marrow hypoplasia rather it is expected to target recipient immune cells residing in non-lymphoid tissues that mediate resistance to donor hematopoietic cell engraftment, and prevent persistent mixed chimerism. Using TLI-svldTBI-ATG recipient conditioning will alter and deplete recipient immune cell compartments and facilitate persistent donor cell chimerism in recipients of living related and unrelated donor organ transplants as well as deceased donor organ transplants of all degrees of HLA mismatch.

The novel TLI-svldTBI-ATG recipient conditioning regimen will be combined with a novel donor hematopoietic cell product that represents a new ‘composition of matter’ for recipients undergoing transplantation on an organ or tissue tolerance protocol from living related and unrelated donors and deceased donors of all degrees of HLA mismatch.

For recipients of living donor organs the hematopoietic cell product will consist of a defined dose of CD34+ cells (a pre-freeze dose of 4-20 million CD34⁺ cells/kg) obtained after short course G-CSF and/or mozobil mobilization with donor apheresis and enrichment by FACS or MACS. The non-CD34+ cell fraction will be used to obtain a defined dose of CD3⁺ T cells (pre-freeze dose of 25-100 million/kg), or selected CD8+ memory T cells (pre-freeze dose of 1-10 million/kg) and/or Treg cells.

The solid organ donor may be living or deceased. In cases of a living donor, hematopoietic cells may be obtained from the solid organ donor using any of the various methods known to one of skill in the art, including apheresis of mobilized peripheral blood from living donors; harvesting hematopoietic cells from bone marrow of deceased donors, and the like. In cases of a deceased donor, hematopoietic cells may be obtained from bone marrow. For example, in a deceased donor the cells may be obtained from the bone marrow in vertebrae, pelvic bone, femur or any other bone or from the spleen which contains sufficient bone marrow from which to extract hematopoietic cells. The unique composition of matter will relate to the ratios of CD34+ cells, CD3+ cells and/or Treg cells that or may not be genetically engineered to express tissue specific chimeric antigen receptors.

### Example 2

Donor hematopoietic cell chimerism and organ transplant tolerance following host conditioning with total lymphoid irradiation (TLI) combined with a single, very low dose of total body irradiation (svldTBI) and anti-thymocyte globulin (ATG) and the infusion of donor CD34+ cells with defined doses of donor CD3+ T cells and/or donor CD8+ memory T cells for transplantation tolerance.

The present example demonstrates the following: 1. Prevent immune mediated rejection of living and deceased donor organ transplants so the graft can survive for the natural life of the recipient. 2. Eliminate or significantly reduce the need for the lifelong requirement of IS drug combinations with their attendant side effects.

Here, we disclose new methods to achieve high levels of persistent mixed chimerism that can be broadly applied to recipients of related and unrelated living, and deceased donor organ (kidney, heart, lung, liver and bowel) transplants that include all degrees of HLA mismatch. This following are described: an improvement to the current TLI-ATG host-conditioning regimen, and a composition to define the ratio of donor CD34+ cells to CD3+ T cells.

When combined together into one protocol the regimen and composition are expected to establish persistent mixed donor cell chimerism in the majority of recipients of related and unrelated living, and deceased donor organ transplants of all degrees of HLA mismatch. regimen without the composition, or vice versa, is unlikely to establish persistent mixed chimerism at levels high enough to support IS drug minimization/withdrawal. The two together are important to achieve success.

High levels (>20% donor T cell chimerism) of persistent mixed chimerism extending beyond one year after organ transplant will support IS drug withdrawal, or IS ‘partial drug withdrawal’ during the second year. The definition of ‘partial drug withdrawal’ will refer to a significant IS drug minimization, defined as a low therapeutic dose of a single IS medication, monotherapy, which is not expected to be associated with the medical co-morbidities caused by current multi-IS drug regimens.

### Regimen

We herein describe a significant improvement and modification to the current TLI-ATG host conditioning regimen that we developed and have used for more than 18 years to induce persistent chimerism in HLA matched living related and unrelated donor transplants patients.

In cancer patients: we reported outcomes of more than 600 cancer patients transplanted using TLI-ATG host conditioning and grafts from HLA matched and mismatched related and unrelated donors. A goal in the cancer patient studies is complete donor chimerism that is required for beneficial graft versus tumor reactions for cancer cures.

In renal tolerance transplant patients: we reported the outcomes of more than 50 patients who received a combined kidney and hematopoietic cell transplant from their living related HLA matched and mismatched donor using TLI-ATG conditioning. In these studies, persistent mixed chimerism was the goal as this allows immune suppression drug withdrawal and discontinuation without organ graft rejection.

The safety profile of TLI-ATG conditioning in all of the above-mentioned studies is well documented: the regimen is low intensity and well tolerated even in patients up to 80 years of age, does not induce severe cytopenias, and is not associated with GI toxicity including mucositis. Host conditioning with TLI-ATG establishes durable donor hematopoietic cell engraftment in HLA matched recipients. The regimen protects against GVHD.

In moving from HLA matched to HLA mismatched donors, and to deceased donors immune mediated resistance to persistent donor hematopoietic cell engraftment will increase. To facilitate persistent mixed chimerism for mmLD-HC and ddVB-BMC we propose a unique improvement and modification to the TLI-ATG regimen in a manner not intuitive nor previously reported.

Host conditioning using a low dose single fraction of TBI alone, or more commonly, in combination with fludarabine and/or alkylating chemotherapy agents has been used for decades in cancer patients undergoing allogeneic hematopoietic cell transplantation. In these studies the dose of TBI ranged from 200-400 cGy which although considered a ‘reduced intensity’ dose induced significant marrow hypoplasia and cytopenias such that virtually all patients developed profound neutropenia, thrombocytopenia, and anemia that required transfusion support for at least 2-3 weeks. Because of the profound host marrow hypoplasia and host immune cell depletion from the single dose of TBI (200-400 cGy) complete donor cell chimerism generally occurred. The TBI (200-400 cGy) based host conditioning regimens are associated with acute GVHD in about 40% of recipients, and chronic GVHD in about 30% recipients.

In the current application, we describe a non-obvious modification, and improvement that is: TLI-ATG will be administered in the regular manner yet instead of 10 doses of TLI (80-120 cGy/dose) one dose of TLI will be omitted, and replaced with a single, yet very low dose of TBI (svldTBI, 40-140 cGy). Currently and despite decades of using TBI host conditioning for cancer patients and organ tolerance regimens, a single TBI dose of less than 200 cGy has not been previously administered, in part, because a single dose less than 200 cGy does not induce marrow hypoplasia to facilitate donor cell engraftment and chimerism.

In the current invention application, the single dose of TBI is novel, and not discussed or mentioned in an aggregate of over 40 years of scientific and medical literature highlighting the use of TBI to support allogeneic hematopoietic cell transplantation. The svldTBI (40-140 cGy) as described herein is not to induce marrow hypoplasia. Rather, the svldTBI will eradicate tissue resident memory T cells residing outside the fields of TLI that mediate resistance to allogeneic donor cell engraftment. Even in the absence of prior exposure to alloantigens, 1-10% of the memory T cells are endogenous alloreactive naturally occurring memory T cells that can react to allogeneic major histocompatibility complex (MHC) molecules in vitro. It is likely that these memory cells are generated through the recognition of peptides from commensal bacteria or environmental antigens presented by self-MHC, which can mimic complexes formed by allogeneic MHC molecules bound to other peptides. Antigen mimicry, named “heterologous immunity,” is well documented in humans and experimental animal models. These naturally occurring alloreactive tissue resident memory T cells mediate resistance to donor hematopoietic cell engraftment and impede the likelihood of achieving persistent mixed chimerism.

It is posited that the replacement of one TLI fraction with a svldTBI will maintain the safety of TLI-ATG, and not increase toxicity owning to the very low single dose of TBI yet will enhance the ability to achieve sustained mixed chimerism when TLI-svldTBI-ATG is combined with a specific composition of matter of the donor cell inoculum.

### Composition

We now describe a novel ‘composition of matter’ for a donor cell product that will support persistent chimerism and allow IS drug minimization and/or cessation following combined organ (kidney, heart, liver, lung, and bowel) and hematopoietic cell transplants from living related and unrelated donors of all degrees of HLA mismatch, and from deceased donors. The donor cell inoculum we describe will protect against GVHD. The donor cell inoculum is specifically paired with TLI-svldTBI-ATG host conditioning, and combined together will support persistent mixed chimerism.

In the case of mmLD-HC (related and unrelated donors): donor hematopoietic cells will be mobilized using granulocyte colony stimulating factor (G-CSF)+/−mozobil, and the donor will undergo 1 or 2 consecutive days of high volume (>12 liters) blood apheresis in the usual manner as per standard of care for BMT donors. The apheresis collection(s) will be processed for CD34+ cell enrichment using either fluorescence activated cell sorting (FAGS) or magnetic activated cell sorting (MACS) as per manufacturer's guidelines. The CD34+ enriched product will be cryopreserved in the standard manner. The pre-freeze CD34+ cell purity must be ≥70%. The CD34+ cell dose will be a pre-freeze value of 10-20 million CD34+ cells/kg recipient weight. The flow through fraction following the MACS or FACS CD34+ enrichment step will be used to obtain a defined dose of CD3+ T cells (a pre-freeze dose of 25-100 million CD3+/kg recipient weight), and will be cryopreserved in the usual manner. When combined together the CD34+ and CD3+ T cell doses are not intuitive, and represent an adjustment through graft manipulation from the naturally occurring physiologic cell populations and ratios. The exact ratio of CD34+ cells to CD3+ cells will depend on the donor (living related versus unrelated) and the degree of HLA antigen mismatch. For example, a living related donor who is 1- or 2-HLA antigen mismatched with the patient the CD34+:CD3+ cell ratio will approximate 1:5. An unrelated living donor who is >3-Ag mismatched with the recipient will approximate a CD34+t CD3+ T cell ratio of 1:10. In all cases of living related or unrelated HLA mismatched donor recipient pairs, the CD34+ and CD3+ cell product ratio is not physiologic or intuitive and is specifically engineered to support persistent donor cell chimerism when combined with the novel host conditioning regimen of TLI-svldTBI-ATG.

In some rare instances (possibly 5 or 6 antigen HLA mismatched unrelated donors) a highly defined donor cell population consisting of CD3+/CD8+/CD45RA−/CD45RO+ T cells (called CD8+ memory T cells) that do not induce GVHD will be obtained from the CD34+ cell flow through fraction instead of CD3+ T cells. The pre-freeze requirements for CD8+ memory T cells will be 1-10 million/kg, with ≥75% purity and viability. This unique cell population, and the methods used to obtain the cells are described in U.S. Pat. No. 9,833,477 B2 which pertained to CD8+ memory T cells possessing graft-versus-tumor (GVT) activity but without GVHD that is important for cancer cures in BMT cancer patients. In the current application, the CD34+ enriched donor cell fraction will be combined with donor CD8+ memory T cells (a novel ‘composition of matter’) at a ratio of 1:1 (range of 1:0.25 to as high as 1:1.5 of CD34:CD8 memory T, respectively) to support persistent mixed chimerism for transplant tolerance following the administration of TLI-svldTBI-ATG host conditioning. This combination of unique cell populations and ‘composition of matter’ is not an intuitive concept based on what is available in the public domain.

In the case of transplants using deceased donor organs: we will obtain deceased donor bone marrow cells (BMC) from vertebral bodies (VBs) as described by others. The bone marrow CD34+ and CD3+ T cell fractions will be enumerated, and cryopreserved in the usual manner. The donor BMC will be infused into the recipients following the host TLI-svldTBI-ATG conditioning. A traditional BMC harvest used in allogenic BMT for cancer patients and described for more than four decades contains a ratio of CD34+ cells to CD3+ cells approximating 1:20. In the current application to induce persistent mixed chimerism in organ transplant recipients for tolerance induction the composition of matter for ddVB-BMC requires a CD34+ cell to CD3+ T cell ratio of about 1:10 (with an upper limit of 1:15). If the absolute number of CD34+ cells is consistently less than the lower limit of 5 million/kg recipient weight needed to establish persistent mixed chimerism, than deceased donor splenocytes will be used to obtain additional CD34+ cells that will be added to the ddVB-BMCs. If the absolute number of CD3+ T cells is consistently less than the lower limit of 40 million/kg recipient weight needed to establish persistent mixed chimerism, then deceased donor splenocytes will be used to obtain and augment the CD3+ T cell dose to fulfill the desired threshold of 40-100 million/kg.

To obtain splenocytes, we previously determined (unpublished data on file) that several (typically 3-to-6) 1-inch sized cubes removed from the donor spleen will be needed. The splenic cubes will be harvested during the time of organ procurement and transported in standard transport media along with the donor VBs. A single cell suspension consisting of live mononuclear splenocytes will be obtained by first dissociating the cells from the tissue in a dissociation media. The expressed cells will be passed through a 100 micron strainer and the cell pellet collected by centrifugation. Using differential centrifugation in Ficoll with or without MACS/FACS separation live mononuclear cells will be obtained. A defined aliquot following this final step will provide splenic CD34+ cells or CD3+ T cells. The splenic CD34+ cells and/or CD3+ T cells will be infused with the ddVB-BMCs to enable persistent donor cell chimerism and support transplantation tolerance. At times, a “left over” fraction of the ddVB-BMC+/−splenocytes may be cryopreserved and stored for months to years, and may be administered as a late donor cell boost if chimerism is waning over time.

### Example 3

Herein describe the novel creation of a deceased donor Tissue Bank consisting of splenic and bone marrow derived hematopoietic stem cells and precursor cell populations, mesenchymal stem cells, dendritic cell populations, stromal cells CD3+ Th1/Th2Th17/Tfh T cells, CD19+ B cells, regulatory T cells (Treg), and invariant natural killer (iNK T cells) for clinical use. It is expected that non-physiologic ratios of sub-sets of deceased donor spleen and bone marrow cells populations will induce organ or tissue transplant tolerance, control refractory and relapsing autoimmune diseases and stimulate therapeutic ‘regenerative medicine’ responses that result in tissue healing and a return to healthier function.

We developed methods to characterize, and enrich a variety of cell populations deceased donor spleen and bone marrow cells that will be cryopreserved for later clinical use. In some cases, for example, we will use a 40-color FACS panel to quantify, characterize, sort and separate cell subsets: The Table below outlines one such approach using 40-color flow to characterize subpopulations of deceased donor spleen and bone marrow cells for cryopreservation and later clinical use.

| | T cells 1 | T cells 2 | T cells 3 | | |
|---|---|---|---|---|---|
| | Cell Subset | | | | |
| | | | Ag Spec | B & NK | |
| Color | Th1/Th2/Th17/Tfh | iNKT + Treg | & Treg | cells | Myeloid 1 |
| 1 | CD45 (USP40) | CD45 (USP40) | CD45 (USP40) | CD45 (USP40) | CD45 (USP40) |
| 2 | CD3 (USP40) | CD3 (USP40) | CD3 (USP40) | CD3 (USP40) | CD3 (USP40) |
| 3 | CD34 (USP40) | CD34 (USP40) | CD34 (USP40) | CD34 (USP40) | CD34 (USP40) |
| 4 | CD19 | CD19 | CD19 | CD19 | CD19 |
| 5 | CD56 | CD56 | CD56 | CD56 | CD56 |
| 6 | CD11b | CD11b | CD11b | CD11b | CD11b |
| 7 | HLA-DR | HLA-DR | HLA-DR | HLA-DR | HLA-DR |
| 8 | L/D Blue | L/D Blue | L/D Blue | L/D Blue | L/D Blue |
| 9 | CD4 | CD4 | CD4 | CD20 | CD1d |
| 10 | CD8 | CD8 | CD8 | IgM | CD16 |
| 11 | 6B11 (iNKT) | 6B11 (iNKT) | 6B11 (iNKT) | IgD | CD141 |
| 12 | g/d TcR | g/d TcR | g/d TcR | IgG | CD303 |
| 13 | CD45RO | CD45RO | CD45RO | CD1d | CD1c |
| 14 | CD62L | CD62L | CD62L | CD22 | CD2 |
| 15 | CD31 | CD31 | CD31 | | CD5 |
| 16 | CD25 | CD25 | CD25 | CD23 | CD81 |
| 17 | CD127 | CD127 | CD127 | CD5 | AXL |
| 18 | KLRG1 | CD95 | FASL/CD178 | CD24 | CD68 |
| 19 | Tim3 | CD94 | CD150 | CD27 | CD273/PD-L2 |
| 20 | CD183/CXCR3 | CD161 | CD73 | CD16 | CD274/PD-L1 |
| 21 | CD185/CXCR5 | CD183/CXCR3 | TIM-1 | CD314 | CD32b |
| | | | | NKG2D | |
| 22 | CD279/PD-1 | CD152/CTLA4 | CD154/CD40L | Tim-1 | CD172a/SIRP1a |
| 23 | TIGIT | CD49b | CD275/ICOS-L | CD244/2B4 | CD88 |
| 24 | CD134/OX40 | CD223/LAG-3 | CD137/4-1BB | NKp46 (CD335) | CD89 |
| 25 | CD27 | CD184/CXCR4 | CD52 | CD71 | CD163 |
| 26 | CD57 | CD314/NKG2D | CD158 | | FceR1a |
| | | | KIR2DL1 | | |
| | | | Clone HP-MA4 | | |
| 27 | Donor HLA | Donor HLA | Donor HLA | Donor HLA | Donor HLA |
| 28 | CD7 | CD7 | CD7 | CD7 | CD7 |
| 29 | CD10 | CD10 | CD10 | CD10 | CD10 |
| 30 | CD38 | CD38 | CD38 | CD38 | CD38 |
| 31 | CD45RA | CD45RA | CD45RA | CD45RA | CD45RA |
| 32 | CD90 | CD90 | CD90 | CD90 | CD90 |
| 33 | CD117 | CD117 | CD117 | CD117 | CD117 |
| 34 | CD135 | CD135 | CD135 | CD135 | CD135 |
| 35 | CD33 | CD33 | CD33 | CD33 | CD33 |
| 36 | CD123 | CD123 | CD123 | CD123 | CD123 |
| 37 | CD14 | CD14 | CD14 | CD14 | CD14 |
| 38 | CD41/61 | CD41/61 | CD41/61 | CD41/61 | CD41/61 |
| 39 | CD66b | CD66b | CD66b | CD66b | CD66b |
| 40 | CD15 | CD15 | CD15 | CD15 | CD15 |
| 41 | CD11c | CD11c | CD11c | CD11c | CD11c |

In some cases, specific precursor and immune cell subsets will be genetically engineered to harbor a unique chimeric antigen receptor that will alter cell trafficking to tissues that include but are not limited to the lung, liver, skin, kidney, vascular endothelium, gut or central/peripheral nervous system. As an example, in some cases, splenic Treg cells will be engineered to express the antigen receptor for Mucosal addressin cell adhesion molecule 1 (MADCAM1) that will direct the engineered splenic Treg cell to the gastrointestinal mucosa to induce site directed tissue healing through enhanced immune regulation. Yet, in other cases, selected spleen and bone marrow cell subsets will be engineered to have synthetic capabilities, with or without engineered antigen receptors, which induce tissue healing through immune regulation, and/or modification of the ECM.

What is claimed is:

1. A method for achieving immune tolerance in a recipient, the method comprising:
conditioning the recipient with a plurality of total lymphoid irradiation doses, and a single dose of very low total body irradiation (svldTBI) of from 40 to 140 cGy;
infusing the recipient with a donor, in vitro engineered, hematopoietic stem cell product;
wherein the recipient achieves stable, high level mixed-chimerism with the donor hematopoietic cells.

2. The method of claim 1, wherein the donor comprises 1 or more MHC-mismatches relative to the recipient.

3. The method of claim 1 or claim 2, wherein the donor comprises 3 or more MHC-mismatches relative to the recipient.

4. The method of any of claims 1-3, wherein the donor is living.

5. The method of any of claims 1-3, wherein the donor is deceased.

6. The method of any of claims 1-5, wherein following the infusion of the hematopoietic stem cell product, the recipient is transplanted with a solid tissue or organ.

7. The method of any of claims 1-5, wherein the recipient has an autoimmune disease.

8. The method of any of claims 1-5, wherein the hematopoietic stem cell product provides for a regenerative medicine benefit.

9. The method of any of claims 1-8, wherein the plurality of total lymphoid irradiation doses comprises a total dose of from 7.2 to 8 Gy, delivered in fractionated doses of 0.8 Gy.

10. The method of any of claims 1-9, wherein one or more doses of ATG are administered to the recipient.

11. The method of any of claims 1-10, wherein the final irradiation dose is the svldTBI dose.

12. The method of any of claims 1-11, wherein the hematopoietic stem cell product has a pre-freeze value of from about 4 to about 20×10⁶ CD34⁺ cells/kg recipient weight.

13. The method of claim 12, wherein the hematopoietic stem cell product has a pre-freeze value of from about 8 to about 100×10⁶ CD3⁺ cells/kg recipient weight, infused from 0 to 3 days following infusion of the CD34⁺ cells.

14. The method of claim 12 or claim 13, wherein the hematopoietic stem cell product has a pre-freeze value of from about 1 to about 12×10⁶ cells/kg donor derived CD8⁺ memory T cells, infused from 0 to 3 days following infusion of the CD34+ cells.

15. The method of claim 14, wherein the CD8+ memory T cells are CD3⁺/CD8⁺/CD45RA⁻/CD45RO⁺ cells.

16. The method of claim 14 or 15, wherein embodiments the CD8+ memory T cells are provided in the place of CD3⁺ cells.

17. The method of any of claims 12-16, wherein the hematopoietic stem cell product has a pre-freeze value of from about 1 to about 10×10⁶ cells/kg Treg cells, infused from 0 to 4 days following infusion of the CD34+ cells.

18. The method of claim 17, wherein the Treg cells are CD4⁺CD25⁺FoxP3⁺ cells.

19. The method of claim 17 or 18, wherein the donor Treg cells are combined with donor CD3+ T cells at a ratio of Treg:CD3+ T cells ranging from 1:50 to 3:1.

20. The method of claim 12 or claim 13, wherein the ratio of CD34⁺ cell to CD3⁺ T cell ratio is from about 1:4 to about 1:15.

21. The method of claim 14, wherein the ratio is about 1:10.

22. An engineered hematopoietic stem cell product having a pre-freeze value of from about 4 to about 20×10⁶ CD34⁺ cells/kg recipient weight.

23. The stem cell composition of claim 22, comprising from about 8 to about 100×10⁶ CD3⁺ cells/kg recipient weight.

24. The stem cell composition of claim 22 or 23, comprising from about 1 to about 12×10⁶ cells/kg donor derived CD8⁺ memory T cells.

25. The composition of claim 24, wherein the CD8+ memory T cells are CD3⁺/CD8⁺/CD45RA⁻/CD45RO⁺ cells.

26. The composition of claim 24, wherein embodiments the CD8+ memory T cells are provided in the place of CD3⁺ cells.

27. The composition of any of claims 22-26, comprising a pre-freeze value of from about 1 to about 10×10⁶ cells/kg Treg cells.

28. The composition of claim 27, wherein the Treg cells are CD4⁺CD25⁺FoxP3⁺ cells.

29. The composition of claim 27 or 28, wherein the donor Treg cells are combined with donor CD3+ T cells at a ratio of Treg:CD3+ T cells ranging from 1:50 to 3:1.

30. The composition of claim 23, wherein the ratio of CD34⁺ cell to CD3⁺ T cell ratio is from about 1:1 to about 1:15.

31. The composition of claim 30, wherein the ratio is about 1:10.
